(19) United States
(12) Patent Application Publication
(10) Pub. No.: US 2016/0110499 A1
(43) Pub. Date: Apr. 21, 2016

(54) METHODS, SYSTEMS, AND COMPUTER-READABLE MEDIA FOR BLIND DECONVOLUTION DEPHASING OF NUCLEIC ACID SEQUENCING DATA

(71) Applicant: LIFE TECHNOLOGIES CORPORATION, Carlsbad, CA (US)

(72) Inventor: Benjamin DONNET, Wynnum (AU)

(21) Appl. No.: 14/886,328

(22) Filed: Oct. 19, 2015

Related U.S. Application Data
(60) Provisional application No. 62/066,405, filed on Oct. 21, 2014.

(51) Int. Cl. G06F 19/22; C12Q 1/68

(52) U.S. Cl. CPC G06F 19/22; C12Q 1/6874

(57) ABSTRACT

Embodiments disclose methods, systems, and computer-readable media for base-calling of sequencing data in the presence of systematic or phasic synchrony errors. These techniques may be adapted to rapidly and accurately resolve signal data arising from a variety of different nucleic acid sequencing platforms. In various embodiments, techniques for blind deconvolution dephasing of nucleic acid sequencing data may be adapted to perform raw signal processing, basecalling, and/or sequence determination.

### CROSS-REFERENCE TO RELATED APPLICATIONS

This application claims priority under 35 U.S.C. §119(e) to U.S. Provisional Application No. 62/066,405, filed Oct. 20, 2014, entitled “Methods, Systems, and Computer-Readable Media for Blind Deconvolution Dephasing of Nucleic Acid Sequencing Data,” and the contents of the foregoing application are incorporated herein by reference in their entirety.

### FIELD OF THE DISCLOSURE

The present disclosure is directed generally to inventive methods, systems, and computer-readable media relating to nucleic acid sequencing, and more particularly, to techniques for improved phase-error correction and base-calling of sequencing data.

### BACKGROUND

Emerging technologies for nucleic acid sequencing and genetic analysis continue to revolutionize many aspects of biology and medicine. Sequencing costs have steadily declined in recent years while the overall instrument throughput has increased dramatically. Current technologies generate data for many nucleic acid (e.g. DNA and RNA) fragments sequenced in parallel. Typically, long strands of genetic material are broken or separated into comparatively short fragments and the nucleotide sequence of the fragments is determined. An important step to elucidate the actual nucleic acid sequence for each sample fragment involves “basecalling.” Basecalling resolves signal information generated by a sequencing instrument and identifies candidate nucleotide or base identifications from the data.

In next generation sequencing technologies, especially those utilizing sequencing-by-synthesis techniques, loss of phase synchrony and systematic noise create difficulties in resolving sequencing signals and can hinder accurate basecalling. It is thus desirable to develop improved analysis techniques capable of processing large amounts of sequence data that are further able to accurately account for noise and phase synchrony effects. A goal of at least certain methods discussed in detail below, among others, is to provide technologies and analysis workflows that efficiently handle and resolve sequencing data in a timely and accurate manner. This goal may be achieved by implementing the processing techniques described below. As a result, the efficiency, accuracy, and speed of data analysis may be substantially improved over existing methods.

### SUMMARY OF THE DISCLOSURE

Embodiments disclose methods, systems, and computer-readable media for accelerated and accurate base calling of sequencing data. These methods may be adapted to improve sequence determination for data arising from a variety of different nucleic acid sequencing platforms. In various embodiments, configurable logic circuits such as FPGAs and GPUs may be adapted to perform raw signal processing, basecalling, and/or sequence determination operations providing further enhancements to the analysis methods. While disclosed in the context of nucleic acid sequencing data resolution and analysis, it will be appreciated that the methods and hardware configurations described herein may be adapted for use with many types of biological data including, for example, DNA, RNA, and protein sequencing signal information.

According to certain embodiments, computer-implemented methods are disclosed for processing raw sequence information or signal data arising from a sequencing instrument. The data may further take many forms determined in part by the type of sequencing technology used. For example, label-based methods for sequence analysis may produce data reflecting signal intensities captured from fluorescent markers or radiolabeled molecules. Alternatively, unlabeled methods for sequence analysis may generate data reflecting detected changes in pH or hydrogen ion concentration.

Analysis of sequencing data typically involves evaluation of instrument sample signal information and performing multiple candidate nucleotide identifications or “basecalls.” Bases or nucleotides may be identified by comparing the likelihood or confidence in various putative identifications. Conventionally, such processes are highly iterative and may take significant computing resources and time to complete. A single sample analysis may require millions, billions, or more of discrete base identifications, each with demanding computational requirements. Inefficiencies in algorithmic design and/or limited computational power of the hardware used to process the signal data can result in performance bottlenecks leading to long processing times and inaccurate results.

According to certain embodiments, improved methods for performing sequence signal processing and basecalls are disclosed. These methods are adaptable to specialized hardware platforms capable of being programmed to operate as dedicated data processing engines with greater efficiency than may be achieved using software running in typical general purpose computing platforms and distributed systems. Such methods may be applied, for example, to raw signal data generated by sequencing instruments performing sequencing-by-synthesis. According to further embodiments, non-transitory computer readable media are disclosed that store instructions that, when executed by a computer, cause the computer to perform blind deconvolution-based methods for base calling of sequencing data.

According to various embodiments, disclosed herein is a method for estimating numbers of nucleotide incorporations that take place during a nucleic acid sequencing-by-synthesis reaction. The method further comprising the steps of: (a) disposing a plurality of template polynucleotide strands in a reaction confinement region disposed on a sensor array, the reaction confinement region containing a sequencing primer and a polymerase; (b) exposing the template polynucleotide strands and the sequencing primer and polymerase to a series of flows of nucleotide species flowed according to a predetermined ordering of nucleotide flows; (c) obtaining a plurality of nucleotide incorporation signals for the reaction confinement region from the sensor array, the nucleotide incorporation signals having an intensity related to a number of nucleotide incorporations having occurred in response to the nucleotide flows; (d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and (e) estimating a number of nucleotide incorporations having occurred for the template polynucleotide strands in the reaction confinement region based on the result of the multiplication converted back to time domain.

According to other embodiments, the present disclosure provides a non-transitory machine-readable storage medium comprising instructions which, when executed and/or implemented by a processor and/or field programmable gate array, cause the processor and/or field programmable gate array to perform a method for estimating numbers of nucleotide incorporations associated with a nucleic acid sequencing-by-synthesis reaction. The method performed by the programmable gate array may further comprise the steps of: (a) disposing a plurality of template polynucleotide strands in a reaction confinement region disposed on a sensor array, the reaction confinement region containing a sequencing primer and a polymerase; (b) exposing the template polynucleotide strands and the sequencing primer and polymerase to a series of flows of nucleotide species flowed according to a predetermined ordering of nucleotide flows; (c) obtaining a plurality of nucleotide incorporation signals for the reaction confinement region from the sensor array, the nucleotide incorporation signals having an intensity related to a number of nucleotide incorporations having occurred in response to the nucleotide flows; (d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and (e) estimating a number of nucleotide incorporations having occurred for the template polynucleotide strands in the reaction confinement region based on the result of the multiplication converted back to time domain.

According to still further embodiments, the present disclosure provides a system, including: a machine-readable memory; and a processor and/or field programmable gate array configured to execute and/or implement machine-readable instructions, which, when executed and/or implemented by the processor and/or field programmable gate array, cause the system to perform a method for estimating numbers of nucleotide incorporations in a nucleic acid sequencing-by-synthesis reaction. The method for estimating nucleotide incorporations may further comprise the steps of: (a) disposing a plurality of template polynucleotide strands in a reaction confinement region disposed on a sensor array, the reaction confinement region containing a sequencing primer and a polymerase; (b) exposing the template polynucleotide strands and the sequencing primer and polymerase to a series of flows of nucleotide species flowed according to a predetermined ordering of nucleotide flows; (c) obtaining a plurality of nucleotide incorporation signals for the reaction confinement region from the sensor array, the nucleotide incorporation signals having an intensity related to a number of nucleotide incorporations having occurred in response to the nucleotide flows; (d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and (e) estimating a number of nucleotide incorporations having occurred for the template polynucleotide strands in the reaction confinement region based on the result of the multiplication converted back to time domain.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the scope of disclosed embodiments, as set forth by the claims.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates schematically a process for sequencing according to exemplary embodiments of the present disclosure;

FIG. 1B depicts a nucleic acid processing system according to exemplary embodiments of the present disclosure;

FIG. 2 depicts a sequencing data analysis workflow according to exemplary embodiments of the present disclosure;

FIG. 3 depicts an exemplary embodiment comprising modules associated with a nucleic acid sequencing workflow.

FIGS. 4A-C illustrate exemplary principals of operation of sequencing signal resolution according to the present disclosure.

FIG. 5 illustrates an exemplary method for blind deconvolution and dephasing of sequencing data according to exemplary embodiments of the present disclosure.

FIGS. 6A-C illustrate exemplary deconvolution processes applied to a sample dataset according to exemplary embodiments of the present disclosure.

FIGS. 7A-B illustrate exemplary results of total energy normalization according to the present disclosure.

FIG. 8 illustrates an exemplary method for nucleotide sequencing signal processing used in connection with Wiener deconvolution methods.

FIGS. 9A-C illustrate metrics resulting from operation of blind deconvolution basecalling methods using an exemplary sequencing signal dataset.

FIGS. 10A-B illustrate an exemplary application of blind deconvolution analysis through several iterations of PSF estimation.

FIG. 11 is a block diagram of a computer, system, and/or server for executing the methods described according to exemplary embodiments of the present disclosure.

### DETAILED DESCRIPTION OF EMBODIMENTS

The following description of various exemplary embodiments is exemplary and explanatory only and is not to be construed as limiting or restrictive in any way. Other embodiments, features, objects, and advantages of the present disclosure will be apparent from the description and accompanying drawings, and from the claims.

In accordance with the disclosure and principles embodied in this application, new methods, systems, and computer readable media for making basecalls by processing and analyzing signal information/data are disclosed. In various embodiments, analysis techniques are provided that allow high-throughput identification of nucleic acid sequences with increased accuracy, speed, and/or efficiency. In particular, the present disclosure enables rapid deconvolution of large amounts of sequence data (for example, in the form of raw signal information) to accurately identify correct basecalls in the presence of systematic errors, signal artifacts, weak incorporation signals and/or other inefficiencies in the underlying sequencing chemistry. The methods described herein are also well suited for resolving basecalls where sequencing effects and artifacts (such as, e.g., phasing effects) are present.

Exemplary embodiments of the present disclosure relate, in part, to the use of large arrays of chemically sensitive field effect transistors (“chemFETs”), and more particularly to ion-sensitive field effect transistors (“ISFETs”). These technologies can detect chemical reactions, including for example nucleic acid (e.g., RNA and DNA) sequencing reactions, by monitoring analytes present, generated, and/or used during a reaction.

Various platforms exist for performing nucleic acid sequencing and each of these platforms generate large volumes of sequencing data. Commercially available sequencing instruments include, the Genome Analyzer/HiSeq/MiSeq platforms (Illumina, Inc.; see, e.g., U.S. Pat. Nos. 6,833,246 and 5,750,341); and the Ion Personal Genome Machine (PGM™) and PROTON™ Sequencer (Life Technologies Corp./Ion Torrent; see, e.g., U.S. Pat. No. 7,948,015 and U.S. Pat. Appl. Publ. Nos. 2010/0137143, 2009/0026082, and 2010/0282617). Sequencing instruments typically generate signal data having particular characteristics and error modes that must be resolved in order to accurately identify the nucleotide bases associated with the signal data. Data generated during a single sequencing run using any next generation sequencing instrument, including those identified above, can produce sequencing signals from independent reactions on the order of millions, tens of millions, or more of that must be quickly and accurately analyzed and resolved. A principal operation in the data analysis workflows for these instruments is processing signal information arising during a given sequencing run and transforming this data into basecalls representative of the underlying template sequence that was detected by the instrument. Before subsequent steps of analysis including fragment alignment, mutation or single polymorphism identification, organism identification, or other clinical and research evaluations may proceed, the transformation of the “raw” sequencing data into base identifications must be accomplished and therefore represents an early gating step in the sequencing workflow.

It will be appreciated that the system and methods described herein may be applied to various instruments, apparatuses, and/or systems for sequencing nucleic acids. Such instruments, apparatuses, and/or systems may include, for example, the Genome Analyzer/HiSeq/MiSeq platforms (Illumina, Inc.; see, e.g., U.S. Pat. No. 6,833,246 and U.S. Pat. No. 5,750,341); the GS FLX, GS FLX Titanium, and GS Junior platforms (Roche/454 Life Sciences; see, e.g., Ronaghi et al., SCIENCE, 281:363 (1998), and Margulies et al., NATURE, 437:376-380 (2005)); and the Ion Personal Genome Machine (PGM™) and PROTON™ Sequencer (Life Technologies Corp./Ion Torrent; see, e.g., U.S. Pat. Appl. Publ. No. 2010/0137143 and No. 2009/0026082, incorporated by reference herein in their entirety).

To increase overall throughput of nucleic acid sequencing, among other objectives, there is a need for new methods, systems, and computer readable media that increase accuracy, speed, and/or efficiency of processing and/or analyzing of large volumes of nucleic acid sequencing data and/or signals. In accordance with the disclosure and principles embodied in this application, new methods, systems, and computer readable media for processing and/or analyzing data and/or signals to accurately resolve high-throughput sequence data are provided. These technologies may further be adapted for use in connection with modified hardware solutions capable of very high speed processing of sequencing data. Implemented in connection with specialized hardware solutions such as FPGA and GPU processors further increases the speed, and/or efficiency of sequence identification relative to conventional general purpose computing software applications.

In this application, “amplifying” generally refers to performing an amplification reaction. In this application, “amplicon” generally refers to a product of a polynucleotide amplification reaction, which includes a clonal population of polynucleotides, which may be single stranded or double stranded and which may be replicated from one or more starting sequences. The one or more starting sequences may be one or more copies of the same sequence, or they may be a mixture of different sequences that contain a common region that is amplified such as, for example, a specific exon sequence present in a mixture of DNA fragments extracted from a sample. Preferably, amplicons may be formed by the amplification of a single starting sequence. Amplicons may be produced by a variety of amplification reactions whose products comprise replicates of one or more starting, or target, nucleic acids. Amplification reactions producing amplicons may be “template-driven” in that base pairing of reactants, either nucleotides or oligonucleotides, have complements in a template polynucleotide that are required for the creation of reaction products. Template-driven reactions may be primer extensions with a nucleic acid polymerase or oligonucleotide ligations with a nucleic acid ligase. Such reactions include, for example, polymerase chain reactions (PCRs), linear polymerase reactions, nucleic acid sequence-based amplifications (NASBAs), rolling circle amplifications, for example, including such reactions disclosed in the following references, which are all incorporated by reference herein in their entirety: Gelfand et al., U.S. Pat. No. 5,210,015; Kacian et al., U.S. Pat. No. 5,399,491; Mullis, U.S. Pat. No. 4,683,202; Mullis et al., U.S. Pat. Nos. 4,683,195; 4,965,188; and 4,800,159; Lizardi, U.S. Pat. No. 5,854,033; and Wittwer et al., U.S. Pat. No. 6,174,670. In an exemplary embodiment, amplicons may be produced by PCRs. Amplicons may also be generated using rolling circle amplification to form a single body that may exclusively occupy a microwell as disclosed in Drmanac et al., U.S. Pat. Appl. Publ. No. 2009/0137404, which is incorporated by reference herein in its entirety.

In this application, “solid phase amplicon” generally refers to a solid phase support, such as a particle or bead, to which is attached a clonal population of nucleic acid sequences, which may have been produced by a process such as emulsion PCR, or like technique, for example.

In this application, “analyte” generally refers to a molecule or biological cell that can directly affect an electronic sensor at a sample retaining region (such as a defined space or reaction confinement region or microwell, for example) or that can indirectly affect such an electronic sensor by a by-product from a reaction involving such molecule or biological cell located in such a sample retaining region. In an exemplary embodiment, an analyte may be a sample or template nucleic acid, which may be subjected to a sequencing reaction, which may, in turn, generate a reaction by-product, such as one or more hydrogen ions, that can affect an electronic sensor. The term “analyte” also comprehends multiple copies of analytes, such as proteins, peptides, nucleic acids, or the like, attached to solid supports, such as beads or particles, for example. In an exemplary embodiment, an analyte may be a nucleic acid amplicon or a solid phase amplicon. A sample nucleic acid template may be associated with a surface via covalent bonding or a specific binding or coupling reaction, and may be derived from, for example, a shot-gun fragmented DNA or amplicon library (which are examples of library fragments further discussed herein), or a sample emulsion PCR process creating clonally-amplified sample nucleic acid templates on particles such as IonSphere™ particles. An analyte may include particles having attached thereto clonal populations of DNA fragments, e.g., genomic DNA fragments, cDNA fragments, or the like.

In this application, “primer” generally refers to an oligonucleotide, either natural or synthetic, that is capable, upon forming a duplex with a polynucleotide template, of acting as a point of initiation of nucleic acid synthesis and being extended from its 3′ end along the template so that an extended duplex may be formed. Extension of a primer may be carried out with a nucleic acid polymerase, such as a DNA or RNA polymerase. The sequence of nucleotides added in the extension process is determined by the sequence of the template polynucleotide. Usually primers are extended by a DNA polymerase. Primers may have a length in the range of from 14 to 40 nucleotides, or in the range of from 18 to 36 nucleotides, for example, or from N to M nucleotides where N is an integer larger than 18 and M is an integer larger than N and smaller than 36, for example. Other lengths are of course possible.

In this application, “oligonucleotide” generally refers to a linear polymer of nucleotide monomers and may be DNA or RNA. Monomers making up polynucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g., naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. In an exemplary embodiment, oligonucleotide may refer to smaller polynucleotides, for example, having 5-40 monomeric units. Polynucleotides may comprise the natural deoxynucleosides (e.g., deoxyadenosine, deoxycytidine, deoxyguanosine, and deoxythymidine for DNA or their ribose counterparts for RNA) linked by phosphodiester linkages. However, they may also comprise non-natural nucleotide analogs, e.g., including modified bases, sugars, or internucleosidic linkages. In an exemplary embodiment, a polynucleotide may be represented by a sequence of letters (upper or lower case), such as “ATGCCTG,” and it will be understood that the nucleotides are in 5′→3′ order from left to right and that “A” denotes deoxyadenosine, “C” denotes deoxycytidine, “G” denotes deoxyguanosine, and “T” denotes thymidine, and that “I” denotes deoxyinosine, and “U” denotes deoxyuridine, unless otherwise indicated or obvious from context.

In this application, “defined space” (or “reaction space,” which may be used interchangeably with “defined space”) generally refers to any space (which may be in one, two, or three dimensions) in which at least some of a molecule, fluid, and/or solid can be confined, retained and/or localized. The space may be a predetermined area or volume, and may be defined, for example, by a depression or a micro-machined well in or associated with a microwell plate, microtiter plate, microplate, or a chip. The area or volume may also be determined based on an amount of fluid or solid, for example, deposited on an area or in a volume otherwise defining a space. For example, isolated hydrophobic areas on a generally hydrophobic surface may provide defined spaces. In an exemplary embodiment, a defined space may be a reaction chamber, such as a well or a microwell, which may be in a chip. A defined space may contain or be exposed to enzymes and reagents used in nucleotide incorporation.

In this application, “reaction confinement region” generally refers to any region in which a reaction may be confined and includes, for example, a “reaction chamber,” a “well,” and a “microwell” (each of which may be used interchangeably). A reaction confinement region may include a region in which a physical or chemical attribute of a solid substrate can permit the localization of a reaction of interest, and a discrete region of a surface of a substrate that can specifically bind an analyte of interest (such as a discrete region with oligonucleotides or antibodies covalently linked to such surface), for example. Reaction confinement regions may be hollow or have well-defined shapes and volumes, which may be manufactured into a substrate. These latter types of reaction confinement regions are referred to herein as microwells or reaction chambers, and may be fabricated using any suitable microfabrication techniques. Reaction confinement regions may be arranged as an array, which may be a substantially planar one-dimensional or two-dimensional arrangement of elements such as sensors or wells.

The number of columns (or rows) of a two-dimensional array may or may not be the same. Preferably, the array comprises at least 100,000 chambers. Preferably, each reaction chamber has a horizontal width and a vertical depth that has an aspect ratio of about 1:1 or less. Preferably, the pitch between the reaction chambers is no more than about 10 microns. Preferably, each reaction chamber is no greater than 0.34 pL, and more preferably no greater than 0.096 pL or even 0.012 pL in volume. Microwells may have any polygonal cross sections, including square, rectangular, or octagonal cross sections, for example, and may be arranged as a rectilinear array on a surface. Microwells may have hexagonal cross sections and be arranged as a hexagonal array, which permits a higher density of microwells per unit area than rectilinear arrays.

A plurality of defined spaces or reaction confinement regions may be arranged in an array, and each defined space or reaction confinement regions may be in electrical communication with at least one sensor to allow detection or measurement of one or more detectable or measurable parameter or characteristics. The sensors may convert changes in the presence, concentration, or amounts of reaction by-products (or changes in ionic character of reactants) into an output signal, which may be registered electronically, for example, as a change in a voltage level or a current level which, in turn, may be processed to extract information about a chemical reaction or desired association event, for example, a nucleotide incorporation event. The sensors may include at least one chemically sensitive field effect transistor (“chemFET”) that can be configured to generate at least one output signal related to a property of a chemical reaction or target analyte of interest in proximity thereof. Such properties can include a concentration (or a change in concentration) of a reactant, product or by-product, or a value of a physical property (or a change in such value), such as an ion concentration.

An initial measurement or interrogation of a pH for a defined space or reaction confinement regions, for example, may be represented as an electrical signal or a voltage, which may be digitalized (e.g., converted to a digital representation of the electrical signal or the voltage). Any of these measurements and representations may be considered raw data or a raw signal. The structure and/or design of sensors for use with the present disclosure may vary widely and may include one or more features of the following references, which are all incorporated by reference herein in their entirety: Rothberg et al., U.S. Pat. Appl. Publ. No. 2009/0127589; Rothberg et al., U.K. Pat. Appl. No. GB24611127; Barbaro et al., U.S. Pat. No. 7,535,232; Sawada et al., U.S. Pat. No. 7,049,645; Kamahori et al., U.S. Pat. Appl. Publ. No. 2007/0059741; Miyahara et al., U.S. Pat. Appl. Publ. Nos. 2008/0286767 and 2008/0286762; O'uchi, U.S. Pat. Appl. Publ. No. 2006/0147983; Osaka et al., U.S. Pat. Appl. Publ. No. 2007/0207471; and Esfandyarpour et al., U.S. Pat. Appl. Publ. No. 2008/0166727.

Arrays including large arrays of chemFETs may be employed to detect and measure static and/or dynamic amounts or concentrations of a variety of analytes (e.g., hydrogen ions, other ions, non-ionic molecules or compounds, etc.) in a variety of chemical and/or biological processes (e.g., biological or chemical reactions, cell or tissue cultures or monitoring, neural activity, nucleic acid sequencing, etc.) in which valuable information may be obtained based on such analyte measurements. Such chemFET arrays may be employed in methods that detect analytes and/or methods that monitor biological or chemical processes via changes in charge at the chemFET surface. Accordingly, at least certain embodiments of the systems, methods, and computer-readable media discussed herein provide uses for chemFET arrays that involve detection of analytes in solution and/or detection of change in charge bound to the chemFET surface.

In this application, “reaction mixture” generally refers to a solution containing all the necessary reactants for performing a reaction, which may include, for example, buffering agents to maintain pH at a selected level during a reaction, salts, enzymes, co-factors, scavengers, and the like, for example.

In this application, “microfluidics device” generally refers to an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, and the like. Microfluidics devices may further include valves, pumps, and specialized functional coatings on interior walls, e.g., to prevent adsorption of sample components or reactants, facilitate reagent movement by electroosmosis, or the like. Such devices are usually fabricated in or as a solid substrate, which may be glass, plastic, or other solid polymeric materials, and typically have a planar format for ease of detecting and monitoring sample and reagent movement, especially via optical or electrochemical methods. Features of a microfluidic device may have cross-sectional dimensions of less than a few hundred square micrometers, for example, and passages may have capillary dimensions, e.g., having maximal cross-sectional dimensions of from about 500 μm to about 0.1 μm, for example. Microfluidics devices may for example have volume capacities in the range of from 1 μL to a few nL, e.g., 10-100 nL.

In various exemplary embodiments, there are provided methods, systems, and computer readable media for processing and analyzing signals and data that allow high-throughput sequencing of nucleic acid sequences with increased accuracy, speed, and/or efficiency. The methods, systems, and computer readable media may include steps and/or structural elements for receiving raw data and/or signals, processing the raw data and/or signals using various protocols and modules, and outputting or storing any results in various formats. In an exemplary embodiment, the results may be further processed or analyzed by other methods, systems, and computer readable media.

In various exemplary embodiments, the methods, systems, and computer readable media described herein may advantageously be used to process and/or analyze data and signals obtained from pH-based nucleic acid sequencing instruments. In pH-based sequencing, a nucleotide incorporation event may be determined by detecting hydrogen ions that are generated as natural by-products of polymerase-catalyzed nucleotide extension reactions. This may be used to sequence a sample or template nucleic acid, which may be a fragment of a nucleic acid sequence of interest, for example, and which may be directly or indirectly attached as a clonal population to a solid support, such as a particle, microparticle, bead, etc. The sample or template nucleic acid may be operably associated to a primer and polymerase and may be subjected to repeated cycles or “flows” of deoxynucleoside triphosphate (“dNTP”) addition (which may be referred to herein as “nucleotide flows”) and washing. The primer may be annealed to the sample or template so that the primer's 3′ end can be extended by a polymerase whenever dNTPs complementary to the next base in the template are added. Then, based on the known sequence of nucleotide flows and on measured signals indicative of hydrogen ion concentration during each nucleotide flow, the identity of the type, sequence and number of nucleotide(s) associated with a sample nucleic acid present in a reaction chamber can be determined.

In pH-based detection methods, the production of hydrogen ions may be monotonically related to the number of contiguous complementary bases in the template strands (as well as the total number of template strands with primer and polymerase that participate in an extension reaction). Thus, when there is a number of contiguous identical complementary bases in the template (i.e. a homopolymer region), the number of hydrogen ions generated is generally proportional to the number of contiguous identical complementary bases. The corresponding output signals may sometimes be referred to as “1-mer”, “2-mer”, “3-mer” output signals, and so on, based on the expected number of repeating bases. Where the next base in the template is not complementary to the flowed nucleotide, generally no incorporation occurs and there is no substantial release of hydrogen ions (in which case, the output signal is sometimes referred to as a “0-mer” output signal).

In each wash step of the cycle, a wash solution (typically having a predetermined pH) is used to remove residual nucleotide of the previous step in order to prevent misincorporations in later cycles. Usually, the four different kinds of nucleotides (e.g. dATP, dCTP, dGTP, and dTTP) are flowed sequentially to the reaction chambers, so that each reaction is exposed to one of the four different nucleotides for a given flow, with the exposure, incorporation, and detection steps being followed by a wash step.

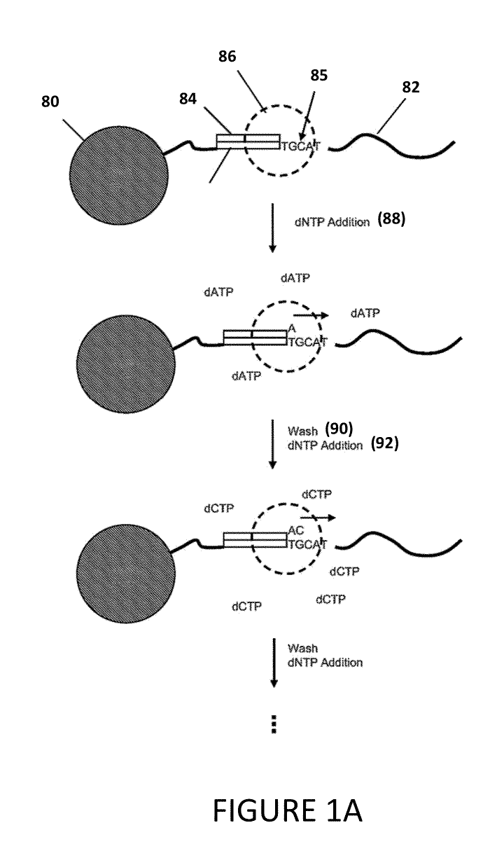

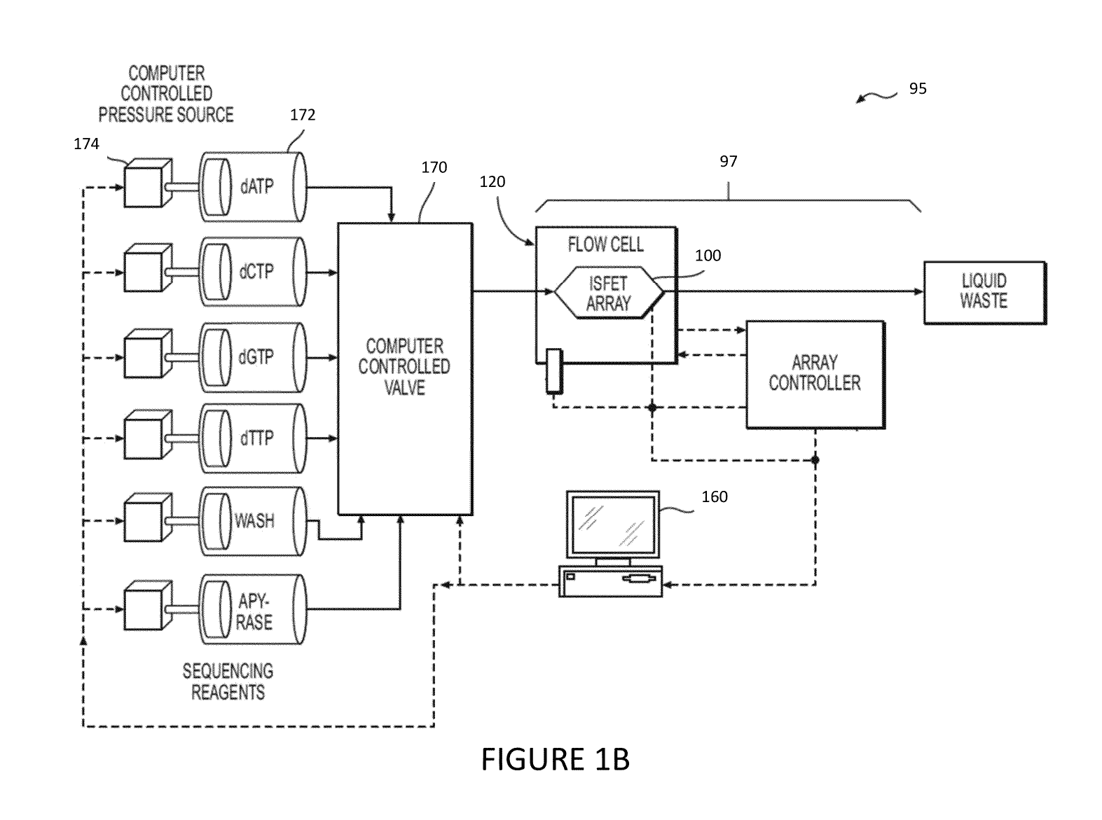

An example of a sequencing-by-synthesis process is illustrated in FIG. 1A, which shows a template polynucleotide strand 82 attached to a particle 80. Primer 84 is annealed to template strand 82 at its primer binding site 81. A DNA polymerase 86 is operably bound to the template-primer duplex. Template strand 82 has the sequence 85, which is awaiting complementary base incorporation. Upon the flow of the nucleotide (shown as dATP), polymerase 86 incorporates a nucleotide since “T” is the next nucleotide in template strand 82 (because the “T” base is complementary to the flowed dATP nucleotide). Wash step 90 follows, after which the next nucleotide (dCTP) is flowed 92. Optionally, after each step of flowing a nucleotide, the reaction chambers may be treated with a nucleotide-destroying agent (such as apyrase) to eliminate any residual nucleotides remaining in the chamber, which can cause spurious extensions in subsequent cycles.

Ideally, each extension reaction associated with a selected population of template polynucleotide strands performs the same incorporation step at the same sequence position in each flow cycle, which can generally be referred to as being “in phase” or in “phasic synchrony” with each other. However, it has been observed that some fraction of template strands in each population may loose or fall out of phasic synchronism with the majority of the template strands in the population. That is, the incorporation events associated with a certain fraction of template strands may either get ahead of or fall behind other template strands in the sequencing run. Such phase loss effects are described in Ronaghi, GENOME RESEARCH, 11:3-11 (2001); Leamon et al., CHEMICAL REVIEWS, 107:3367-3376 (2007); Chen et al., International Patent Publication WO 2007/098049. Such phasing effects can introduce noise into the signal and hinder the ability to make accurate base calls from the signals.

As mentioned above, one cause of phase synchrony loss is the failure of a sequencing reaction to incorporate the same nucleotide at substantially the same time into the growing extension strand associated with a clonal population of template strands. For a given flow cycle this effect may result in some amount of the extending strands falling behind the main population with respect to a particular sequence position. This effect is referred to as an “incomplete extension” error. Another cause of phase synchrony loss is the improper incorporation of one or more nucleotide species in a portion of the extending strands associated with a clonal population of template strands. This effect may result in a portion of the extending strands progressing ahead of the main population in sequence position. This is referred to as a “carry forward” error. Carry forward errors may result from the misincorporation of a nucleotide species, or in certain instances, where there is incomplete removal of a previous nucleotide species in a reaction well (e.g. incomplete washing of the reaction well). Thus, as a result of these phasing effects, at a given flow cycle, the population of template strands may be a mixture of strands in different phase-states. As noted above, the out-of-phase template strands can introduce noise into the signal, making accurate base calling more difficult.

US Patent Application US20140222399 (Ser. No. 14/150,855) entitled “Predictive Model for Use in Sequencing-by-Synthesis” to Davey et al. describes an approach to identify signal correction parameters that include predicting values for phasing effects. Attempting to determine phasing signal correction parameters that can be applied to the signal analysis used for base calling can be difficult and computationally expensive. According to exemplary embodiments of the present disclosure, a process for blind deconvolution and point spread function estimation is applied rather than attempting to determine phase signal correction parameter(s) directly.

FIG. 1B depicts a nucleic acid processing system including a large scale chemFET array, according to exemplary embodiments of the present disclosure. The chemFET sensors of the array are described for purposes of illustration as ISFETs configured for sensitivity to static and/or dynamic ion concentration, including but not limited to hydrogen ion concentration. However, it should be appreciated that the present disclosure is not limited in this respect, and that in any of the embodiments discussed herein in which ISFETs are employed as an illustrative example, other types of chemFETs may be similarly employed in alternative embodiments. Similarly, it should be appreciated that various aspects and embodiments of the present disclosure may employ ISFETs as sensors yet detect one or more ionic species that are not hydrogen ions.

The system 95 may include a semiconductor/microfluidics hybrid structure 97 comprising an ISFET sensor array 100 and a microfluidics flow cell 120. The flow cell 120 may comprise a number of wells (not shown) disposed above corresponding sensors of the ISFET array 100. The flow cell 120 may be configured to facilitate the sequencing of one or more identical template nucleic acids disposed in the flow cell via the controlled and ordered introduction to the flow cell of a number of sequencing reagents 172 (e.g., dATP, dCTP, dGTP, dTTP (generically referred to herein as dNTP), divalent cations such as but not limited to Mg2+, wash solutions, and the like.

As illustrated in FIG. 1B, the introduction of the sequencing reagents to the flow cell 120 may be accomplished via one or more valves 170 and one or more pumps 174 that are controlled by a computer 160. A number of techniques may be used to admit (i.e., introduce) the various processing materials (e.g., solutions, samples, reaction reagents, wash solutions, and the like) into the wells of such a flow cell. As illustrated in FIG. 1B, reagents including dNTP may be admitted to the flow cell (e.g., via the computer controlled valve 170 and pumps 174) from which they diffuse into the wells, or reagents may be added to the flow cell by other means such as direct injection. In yet another example, the flow cell 120 may not contain any wells, and diffusion properties of the reagents may be exploited to limit cross-talk between respective sensors of the ISFET array 100, or nucleic acids may be immobilized on the surfaces of sensors of the ISFET array 100.

The flow cell 120 in the system of FIG. 1B may be configured in a variety of manners to provide one or more analytes (or one or more reaction solutions) in proximity to the ISFET array 100. For example, a template nucleic acid may be directly attached or applied in suitable proximity to one or more pixels of the sensor array 100, or in or on a support material (e.g., one or more “beads”) located above the sensor array but within the reaction chambers, or on the sensor surface itself. Processing reagents (e.g., enzymes such as polymerases) may also be placed on the sensors directly, or on one or more solid supports (e.g., they may be bound to the capture beads or to other beads) in proximity to the sensors, or they may be in solution and free-flowing. It is to be understood that the device may be used without wells or beads.

In the system 95 of FIG. 1B, according to one embodiment the ISFET sensor array 100 monitors ionic species, and in particular, changes in the levels/amounts and/or concentration of ionic species, including hydrogen ions. The species may be a result from a nucleic acid synthesis or sequencing reaction.

Various embodiments of the present disclosure may relate to monitoring/measurement techniques that involve the static and/or dynamic responses of an ISFET. It is to be understood that although the particular example of a nucleic acid synthesis or sequencing reaction is provided to illustrate the transient or dynamic response of chemFET, such as an ISFET, the transient or dynamic response of a chemFET, such as an ISFET, as discussed below may be exploited for monitoring/sensing other types of chemical and/or biological activity beyond the specific example of a nucleic acid synthesis or sequencing reaction.

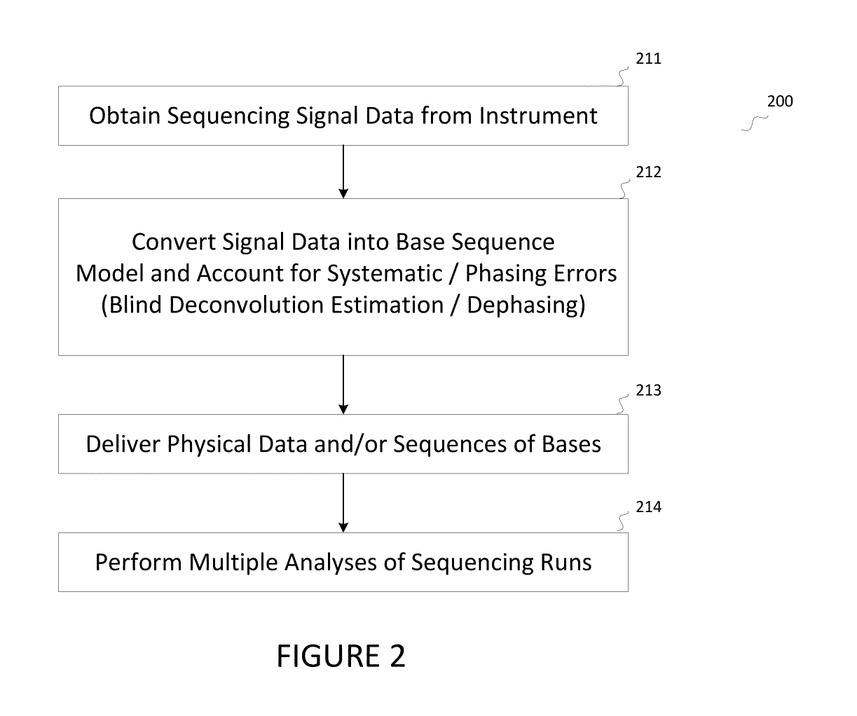

Sequencing platforms typically apply a data analysis workflow that takes into account the characteristics and parameters required to analyze the sequencing signals arising from the instrument. As one example, the Ion Torrent platform (ThermoFisher Scientific Inc., Carlsbad, Calif.) may be configured to perform a sequencing data analysis workflow according to the exemplary embodiment of FIG. 2. Additional details of related methods may be found in US Patent Publication 2013/0090860 (Ser. No. 13/645,058) to Sikora et al. which is incorporated by reference in its entirety.

According to this method 200 sample nucleic acids are evaluated in a series of steps that include making base calls by processing and/or analyzing nucleic acid sequencing data. In step 211, physical data is obtained by performing a sequencing task using a sequencing instrument. In the case on the Ion Torrent platform, the physical data may include voltage or current data indicative of hydrogen ion concentrations. Nucleotide incorporation events are determined, in part, by detecting increases in hydrogen ion concentration. The signal data arising from nucleotide incorporation events detected as increased hydrogen ion concentrations require processing of the signal data.

In step 212, a server or other computing means or resource may be used to convert the signal data into base sequence information by, for example converting the signal data into a sequence of bases by signal modeling and estimating or accounting for systematic and/or phase synchrony effects. According to the present disclosure, and in various embodiments, such signal modeling may include applying a blind deconvolution estimation approach where the sequencing signal data is evaluated to account for systematic errors and/or phase synchrony effects that may take place during sequencing. A candidate base sequence may further be identified as that most likely or most closely fit to the observed signal data. In step 213, the physical data and/or sequences of bases associated with one or more samples may be delivered by the server or other computing means or resource to an end user, saved, or passed to other computing devices or programs for further processing. In step 214, if multiple runs of physical data and/or sequences of bases have been conducted further analyses and comparisons of sequencing run data may be performed.

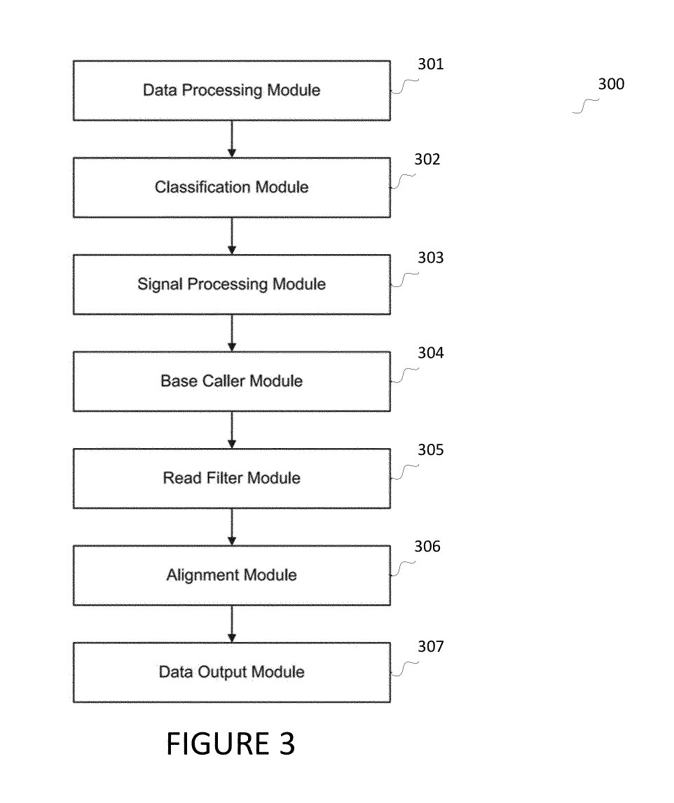

The steps of evaluating the signal data and performing basecalling or sequence identification involve detailed calculations that are performed in connection with each base sequenced for each sample template. These steps may further be performed by a selected nucleic acid sequencing module that is included in a sequence analysis system workflow such as that exemplified in FIG. 3 and described in greater detail hereinbelow.

In FIG. 3, the exemplary embodiment of a nucleic acid sequencing workflow 300 includes a data processing module 301, a classification module 302, a signal processing module 303, a base caller module 304, a read filter module 305, an alignment module 306, and a data output module 307. The sequencing system or platform may be implemented in one or more computers and/or servers and may be accessible at least in part through a web-accessible data portal. In an exemplary embodiment, there is provided a method performing steps including the general steps associated with modules 301-307 (e.g., processing data, classifying defined spaces or reaction confinement regions, processing signals, calling bases, filtering reads, aligning reads, and outputting results).

In an exemplary embodiment, a data processing module or data processor 301 may be configured to receive data (e.g., raw sequencing data, which may comprise a series of signals arising from various samples contained in wells), reflective or indicative of one or more by-product(s) of a chemical reaction. The signals may be derived from nucleotide incorporation events (e.g., incorporation of a dNTP associated with a sample nucleic acid template) by measuring hydrogen ions generated as by-products of polymerase-catalyzed nucleic acid extension reactions. The hydrogen ion concentration (or pH) for a defined space or reaction confinement region may be measured repeatedly and at intervals timed to coincide with the nucleotide flows of different types of dNTPs. The signals may be actual raw pH values, or they may be a conversion of the raw pH value (or related physical measurement) in each defined space into a voltage or current, for example, which may then be converted into a digital representation.

The data processing module 301 may be configured to generate one or more acquisition file(s) for the raw data, which may contain raw signals from defined spaces of a chip, for example, for one or more nucleotide flow(s). For a chip containing about 1.5 million wells, for example, each nucleotide flow can result in about 1.5 million separate nucleotide incorporation events, and a series of such acquisition files can represent about 1.5 million possible reads. A read can represent consecutive base calls associated with a sequence of a nucleic acid. A read can reflect bases or base complements associated with a sample nucleic acid template, which can be associated with a defined volume, such as a well, or with a defined area, such as a portion of a surface of a substantially flat substrate, for example. A read can include a full sequence of the sample nucleic acid template or a portion thereof. A read can include about eight nucleotides (base calls) and can contain 16 or more base calls, 25 or more base calls, 50 or more base calls, 100 or more base calls, or 120 or more base calls, for example. The length of a read can be expressed as a number of base pairs (bps).

In an exemplary embodiment, the data processing module 301 may be configured to perform multiple functions, including receiving or loading raw data and/or signals (which may be temporarily or permanently stored in a memory and may be compressed and decompressed as desired), decompiling raw data, and offset correcting raw data. For example, the raw data and/or signals may be streamed off of an analytical instrument directly to the data processing module. Alternatively, or in combination with direct steaming, the data processing module may access or receive the raw data and/or signals after storage or collection on a computer-readable medium, such as a portable disk or hard drive, for example. The data processing module may receive directly raw acquisition files in DAT file format (e.g., acq_*.dat files), for example, streaming from an analytical instrument.

In an exemplary embodiment, the data processing module 301 may be configured to compress data and/or signals using one or more compression modes, which may include a dynamic/variable frame rate compression mode and a key frame and/or delta compression mode. In the dynamic/variable frame rate compression mode, certain portions of a nucleotide incorporation event or a nucleotide flow may be captured at different frame rates to allow capture of biologically specific events at high resolution while reducing the overall file size by allowing multiple frames in some portions to be averaged. In the key frame and/or delta compression mode, whereas an initial value is actually stored, for subsequent values only their difference relative to the initial value may be stored.

In an exemplary embodiment, the data processing module 301 may be configured to perform raw signal offset and/or background corrections. Each defined space may have its own reference value. To compare two defined spaces, a common reference may be used. The offset and/or background correction can take the average of the first few frames within each acquisition file, and subtract that value from values for each defined space, thus allowing measurements within the defined space to have a common reference value.

In an exemplary embodiment, the data processing module 301 may flag or exclude certain defined spaces that may for whatever reason not be functional or may be covered, obscured, or otherwise fluidically inaccessible or unaddressable. For example, a mask may be loaded, per chip type, to mark those defined spaces as excluded so as to avoid unnecessary and/or computationally inefficient downstream processing of the chip and signals generated therefrom, where the information likely will be uninformative.

In an exemplary embodiment, a classification module or classifier 302 may be configured to classify one or more wells of an array as to whether the well is empty or contains an analyte or substrate associated with an analyte and whether the well generally contains useful information that should be carried forward and included in downstream processing and/or analysis. Because the data can include signals from thousands to millions of individual wells, reducing the amount of data to be carried forward can increase overall performance and efficiency, and conserve file storage space. (Of course, in practice while some data can be screened, all data may be stored so that various screening and manipulating of the data can be started anew, if desired.) The classification module 302 may process wells in smaller groups or regions rather than as one group to exploit parallel computing techniques, such as multi-core and/or multi-process nodes that have parallel computational capabilities. For example, a chip containing an array of about 1.5 million wells can be segmented into 50×50 well regions, resulting in about 625 total regions.

In an exemplary embodiment, the classification module 302 may be configured to classify one or more wells of an array as to whether the well is empty or contains an analyte or substrate associated with an analyte by flowing a known pH buffer at a different pH than a wash buffer onto the wells. If the diffusion rate in the well is slower than an average rate of surrounding neighbors, for example, then the well may be considered to contain a particle. If not, then the well may be identified as empty. Other procedures to establish a baseline pH change over time can include, for example, fitting the signal to exponentials or other models of the expected background signal.

According to an exemplary embodiment, there is provided a method for determining whether a defined space includes an analyte or substrate associated with an analyte, including: (1) changing reagents in a flow chamber from a first reagent that sensors generate in response thereto a first output signal to a second reagent that sensors generate in response thereto a second output signal; and (2) correlating a time delay in the generation of the second output signal in response to the changing of reagents with a presence or absence of an analyte or substrate associated with an analyte. The sensor may be an electrochemical sensor, including a potentiometric sensor, an impedimetric sensor, or an amperometric sensor, for example, or any sensor such that the output signal depends on an interaction between an electrode or other analyte-sensitive surface and a sensor-active reagent whose arrival is delayed by physical or chemical obstructions in a defined space. The sensor-active reagent may be a wash solution at a different pH than the reagent it replaces.

In an exemplary embodiment, the classification module 302 may be further configured to identify and parse sample nucleic acids or fragments based on their type and/or origin. Such identification, which may be useful when using test nucleic acid fragments as a control and/or when pooling and sequencing fragmented samples of nucleic acids from different origin (“multiplexing”), for example, may be based on labeling or tagging of the fragments prior to the sequencing process (e.g., with fluorescent tags). Such identification may be performed using sequencing keys (e.g., a known artificial nucleic acid sequence).

In an exemplary embodiment, a signal processing module or signal processor 303 may be configured to analyze signal information from a defined space or reaction confinement region and an associated sample nucleic acid template. The signal processing module may output a processed signal, which may be considered a raw incorporation signal. The signal processing module 303 may use information and data resulting from the classification module 302 and associated methods, but may also use raw data or raw signals.

In an exemplary embodiment, the signal processing module 303 may be configured to remove noise from raw signal and improve a quality of the signal, which may include an accuracy and a signal-to-noise ratio of the raw signals, for example. Noise, which may be due to various causes including thermal sensitivity of the sensors, electrical potential disturbances in the fluid (such as resistive or thermal noise in the fluids, reference voltage changes due to different fluids contacting the reference electrode), pH changes due to bulk changes in fluids that are passed over the sensor array (referred to herein as “reagent change noise”), stochastic behavior of polymerase function (e.g., incomplete extensions) or failure to completely wash away all dNTPs in a given step (e.g., inappropriate incorporation), for example, may be removed in various ways.

In an exemplary embodiment, the signal processing module 303 may be configured to remove from the data and signals it received some background signal or noise to generate an improved incorporation signal. To minimize computation time, the signal processing module may only process data and signals for defined spaces containing particles and/or having produced a sufficiently strong signal to indicate a nucleotide incorporation event. The background or noise portion of the signal can be present during each flow and can vary over time, across an array of wells, and during an acquisition.

In an exemplary embodiment, the signal processing module 303 may be configured to create an incorporation fitting model, which may have two parts. The first part may include determining the background signal that would have been measured in a given defined space had no nucleotide incorporation event occurred. The second part may include subtracting or otherwise removing (or fitting) the background signal from the raw signal and then examining and analyzing (or fitting) the signal that remains. The result of the incorporation fitting model may be an estimate of incorporation during each nucleotide flow for each well.

In an exemplary embodiment, the signal processing module 303 may be configured to perform or implement one or more of the teachings disclosed in Rearick et al., U.S. patent application Ser. No. 13/339,846, filed Dec. 29, 2011, based on U.S. Prov. Pat. Appl. Nos. 61/428,743, filed Dec. 30, 2010, and 61/429,328, filed Jan. 3, 2011, and in Hubbell, U.S. patent application Ser. No. 13/339,753, filed Dec. 29, 2011, based on U.S. Prov. Pat. Appl. No. 61/428,097, filed Dec. 29, 2010, which are all incorporated by reference herein in their entirety.

In an exemplary embodiment, the signal processing module 303 may receive a MASK file from the classification module 302. The signal processing module 303 may store, transmit, and/or output raw incorporation signals and related information and data in raw WELLS file format, for example. The signal processing module 303 may output a raw incorporation signal per defined space and per flow, for example.

In an exemplary embodiment, a base caller module or base caller 304 may be configured to transform a raw incorporation signal into a base call and compile consecutive base calls associated with a sample nucleic acid template into a read. A base call refers to a particular nucleotide identification (e.g., dATP (“A”), dCTP (“C”), dGTP (“G”), or dTTP (“T”)). The base caller module may perform one or more signal normalizations, signal phase and signal droop (e.g., enzyme efficiency loss) estimations, and signal corrections, and it may identify or estimate base calls for each flow for each defined space. The base caller module may share, transmit or output non-incorporation events as well as incorporation events.

In an exemplary embodiment, the base caller module 304 may be configured to normalize a read, which may include initially using raw data and/or signals from the signal processing module. For example, using one or more known expected 1-mer events, which may be identified using sequencing keys, a 1-mer average signal may initially be established and used for normalization. Then, as the base caller module processes each defined space, additional base calls can be accurately determined and additional measurements then can be used to re-normalize the raw signals. Such re-normalization process may improve confidence (e.g., a higher signal-to-noise ratio) of the signal from each defined space.

In an exemplary embodiment, the base caller module 304 may be configured to observe and account for signal droop that in some instances may be attributed to DNA polymerase loss that can occur during a sequencing run. Such DNA polymerase loss may be experienced during nucleotide incorporation events, with values typically in the range of about 0.1% to about 0.2% over the course of a run. By averaging groups of reads in a region together and/or averaging their signals after normalization, an exponential can be fit to the resulting curve, from which the rate of signal loss over time can be extracted to determine an estimate of the DNA polymerase loss during nucleotide incorporation events. In an exemplary embodiment, the base caller module 304 may be configured to use the signal droop in a signal phase model as a constant for a read. Signal estimates can vary across an array of defined spaces, but signal droop estimates often can be assumed to be fixed for each processed region.

In various embodiments, the methods for blind deconvolution basecalling described herein provide an alternative and effective approach to phase signal estimation and dephasing of sequencing signal data. Additionally, the methods provide useful approaches to resolving sequencing signals associated with sample templates containing homopolymer sequences. During sequencing, signals associated with homopolymers may be difficult to resolve by conventional methods. In particular, as the length of a homopolymer increases, the detected signal may not maintain a linear response relative to other shorter and longer homopolymers. According to the present disclosure, the methods for blind deconvolution provide the ability to deconvolve an observed sequencing signal (which may contain noise and systematic errors) and derive an expected or projected sequencing that may be used to improve basecalling accuracy. Further, such methods may improve the confidence in distinguishing sequencing signals associated with homopolymers by performing basecalling using the expected or projected sequencing signal.

In an exemplary approach, an estimated point spread function (PSF) may be associated with or derived from the sequence signal data for a selected nucleotide flow and observed nucleotide incorporation event. By deconvolving the observed signal and applying the point spread function to the estimated homopolymers, an estimation of signal error may be obtained. Signal errors may result from systematic errors and noise including phase synchrony (e.g. dephasing) errors that result from carry-forward and incomplete extension effects that takes place during nucleic acid sequencing. Such errors tend to accumulate over the course of the sequencing run and degrade the sequence data quality or basecall accuracy if not accounted for. The methods described herein desirably provide mechanisms by which such errors may be accounted for and the underlying sequence data resolved in the presence of such errors. Additional information describing polymerase-based carry-forward and incomplete extension phenomena may be found in Margulies et al., NATURE, 437:376-380 (2005). Phasing effects as described herein may also be referred to as plus frame shifts and minus frame shifts, see, e.g., Ronaghi et al., GENOME RESEARCH, 11:3-11 (2001), plus-shift effects and minus-shift effects, see Svantesson et al., BIOPHYSICAL CHEMISTRY, 110:129-145 (2004), and extension failures, see U.S. Pat. No. 7,875,440.

According to the present disclosure, basecalling can be accurately conducted in the presence of the aforementioned errors and phenomena by iterative association of point spread functions with the observed sequencing data. Distinguished from conventional methods that may attempt to estimate and remove these errors from the sequencing signal data prior to basecalling, the present methods accommodate direct and accurate basecalling in the presence of noise or error-containing data. In an exemplary embodiment, a solver module that applies blind deconvolution basecalling may be configured as a software tool or application with functionality to efficiently solve or determine a candidate sequence that is consistent with observed data. According to the present disclosure, the solver functionality may be improved through the implementation of modified basecalling methods that are suitable for adaptation on dedicated hardware processors. Various embodiments of the architecture and processing of sample sequence signal data will be described in greater detail hereinbelow.

In various embodiments, the sequence identification approaches of the present disclosure may go beyond “base-by-base” base calling, and extend to “whole-sequence” or “whole-read” calling (or at least “whole-fragment” calling). That is, the output of a basecall solution as described herein may be a particular sequence that was collectively considered/called, rather than merely a sequence of individual bases that were considered/called one by one and then joined together to form a sequence. As will be described in detail below, the present methods are distinguished from other basecall determination methods such as the “Treephaser” methods described in Sikora et al., U.S. Patent Application Publication Nos. 2013/0060482 and 2013/0090860 by a number of beneficial enhancements giving rise to improved performance and accuracy.

In various embodiments, the present disclosure dynamically adapts a phasing solution to one or more nucleotide flows under consideration to improve basecalling. PSF kernel optimization techniques are applied to nucleotide flows under consideration and provide the ability to evaluate one or more locally “optimal” solutions for portions of a sample sequence. These solutions may be applied across the entirety of the sample sequence and further may be performed in parallel for one or more different sample sequence templates for which sequencing signals have been generated. In various aspects, prior knowledge of phasing dynamics and/or detailed understanding of other systematic errors is not required to apply the basecalling methods. In one exemplary application, a PSF kernel optimization technique is used to make “blind” estimates for a local solution based on observed sequencing data signals. A window of flows under basecalling consideration may then be re-called using the local solution with iterative progression of the window forward in flow space. This approach allows ambiguous or low confidence basecalls from earlier flows to be corrected and/or updates as new PSF kernel information becomes or is made available. In various embodiments, the PSF may be updated when a phasing event is detected, rather than propagating with each flow.

In various embodiments, the basecall methods may be implemented on a basecall processor comprising one or more dedicated hardware components configured to receive sequencing signal information generated by the sequencing system or instrument. The hardware components may further comprise a programmable FPGA or GPU board configured to process the sequencing signal information according to the methods described herein. In various exemplary embodiments, the FPGA board may comprise a commercially available programmable product such as the Stratix V FPGA (Altera Corporation, San Jose, Calif.). Information regarding field programmable gate array (“FPGA”) technology and/or graphics processing unit (“GPU”) technology may be obtained from the following documents which are incorporated by reference herein in their entirety: Woods, R., et al., FPGA-based Implementation of Signal Processing Systems, John Wiley & Sons (2008); Gallagher, S., Mapping DSP Algorithms Into FPGAs, Xilinx, Inc., available at http://www.ieee.li/pdf/viewgraphs/mapping_dsp_algorithms_into_fpgas.pdf; and Bartholomä, R., et al., Implementing Signal Processing Algorithms on FPGAs, University of Applied Sciences Pforzheim, Germany, available at http://citeseerx.ist.psu.edu/viewdoc/download?doi=10.1.1.130.8731&rep=repl&type=pdf.

In various embodiments, the basecall processor may be configured to receive signal information representative of data obtained from the sequencing platform or instrument. The basecall processor may be embedded or associated directly with the sequencing instrument or reside in a separate computing instrument or platform configured to receive sequence signal information transmitted directly from the sequencing instrument or process the sequencing signal information stored in a file or other data structure. In various embodiments, the basecall processor is configured to independently or semi-independently process the sequence signal information directly thereby freeing up computing resources associated with other sequence analysis systems or subsystems such that they are not encumbered or substantially slowed down during the processing of the sequence signal information.

In the context of next generation, high throughput sequence analysis conventional methods for basecalling may employ probabilistic or machine-learning models. These methods may further require complex statistical-models be developed that require significant understanding of the underlying data characteristics and error modes. Additionally, such approaches may require large amounts of training data to develop sufficient confidence in basecall prediction algorithms. Additionally, such approaches may be computationally inefficient with higher order time complexities. As read lengths for sample sequences increase, degradation in signal quality, loss of phasic synchrony, and increased systematic noise further reduce the performance of conventional approaches. Unlike such approaches, the methods of the present disclosure consider the basecalling problem from a perspective of resolving the underlying sequencing signal in the presence of noise rather than attempting to subtract or remove the noise components of the signal.

Blind deconvolution methods have been applied in the context of optical signal processing for photographic images to discern and recover from a blurred optical signal an underlying component signal that gives rise to the resultant image. Briefly, blind deconvolution methods in an optical context attempt to recover a sharp or highly resolved version of an input image that is obscured by an unknown or poorly characterized blur function (or blur kernel). Mathematically, an idealized description of the image deconvolution problem may be set forth as decomposing a blurred image “y” as y=k\textcircled{x}x where x is a possible unknown sharp image, and k is a non-negative blur kernel, with weak support in comparison to the image size. This problem may give rise to an intractable solution set where an infinite set of pairs (x, k) explain any observed y.

It is therefore desirable that additional assumptions on x or k be introduced to improve the tractability of solution determination. A more refined description of the problem may take the form of y=k\textcircled{x}x+n where the additional component “n” reflects an independent and identically distributed (i.i.d.) Gaussian noise function. A goal of blind deconvolution is to therefore infer both k and x given a single input y. For additional details regarding blind image deconvolution theory see for example Campisi P. & Egiazarian K “Blind image deconvolution: theory and applications” CRC Press (2001) and Kundur D. & Hatzinakos D. “Blind image deconvolution” IEEE Signal Processing Magazine (1996).

As will be described in greater detail hereinbelow, a novel implementation for blind deconvolution signal processing may be applied in the context of non-optical sequencing data. For example, sequencing data obtained from a nucleic acid sequencing instrument such as the Ion Torrent sequencer (ThermoFisher Scientific, Carlsbad, Calif.) which uses chemFET sensors to detect hydrogen ion concentrations generates non-optical data that is subject to phase-synchrony and systematic noise effects. As will be described in greater detail, this data may be processed by blind deconvolution resolution methods applied to the non-optical signal data to facilitate basecalling by identifying an underlying contributing signal (e.g. a sharp signal). Such signals may desirably be discerned in the presence of phase-synchrony and systematic noise effects that mask or obscure the signal.

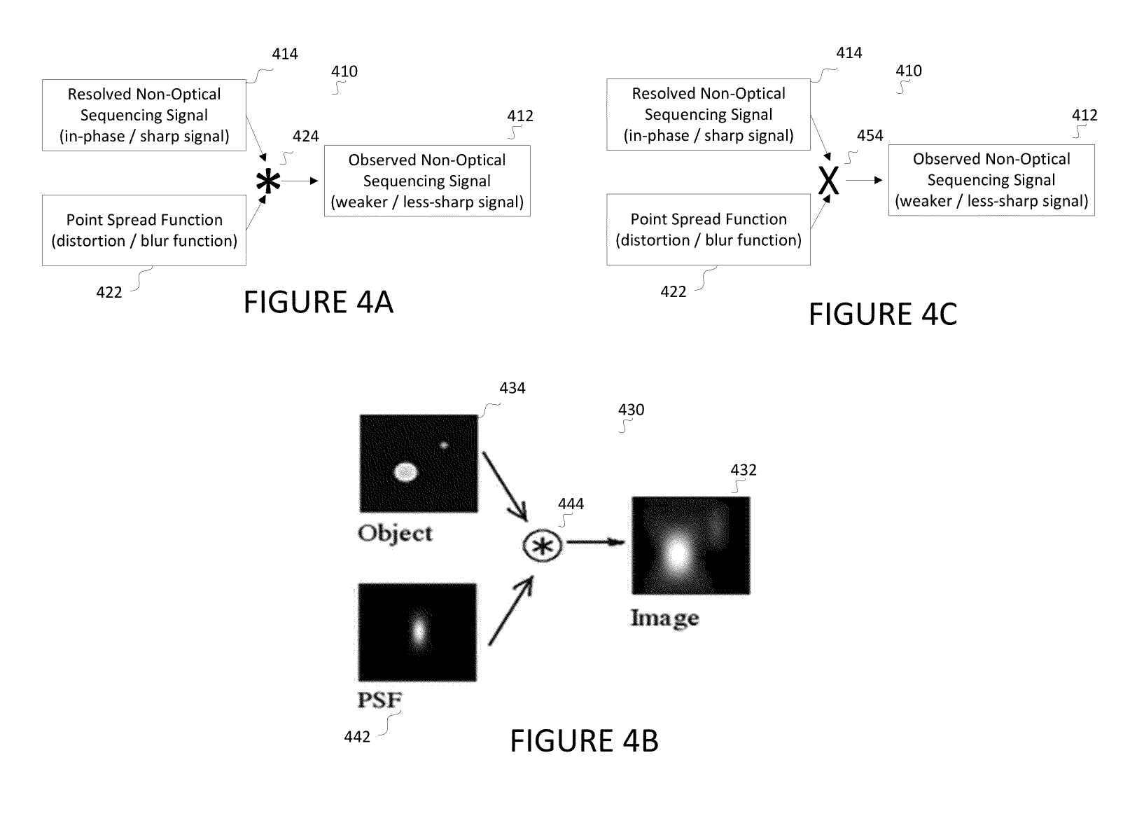

FIGS. 4A-C further illustrate the principals of operation of sequencing signal resolution according to the present disclosure. FIG. 4A describes an exemplary representation of non-optical sequencing signals 410, generated for example by a semiconductor sequencing-by-synthesis technology or platform and compared to an exemplary optical image resolution 430 representation (FIG. 4B). In various embodiments, as shown in FIG. 4A, an observed non-optical sequencing signal 412 includes systematic noise and/or is subject to phasic synchronic errors (analogous to blurry signal 432) that may distort or obscure an underlying or associated idealized sequencing signal 414 (analogous to sharper/in-focus signal 434). The error and noise components of the observed sequencing signal 412 obscure the underlying optimal signal 414 and as a result identifying with a high degree of confidence a likely basecall associated with the sequencing signal is confounded without addressing these sources of noise and error.

In various embodiments, the idealized sequencing signal 414 may represent a portion of the observed sequencing signal 412 that is in-phase (e.g. phasic synchrony contribution) or representative of the non-noise contributions and/or non-out-of-phase signal contributions to the observed sequencing signal 412. A point spread function (PSF) 422 applied to the resolved non-optical sequencing signal 414 by the convolution operation 424 gives rise to the observed non-optical sequencing signal 412 (analogous to an optical image PSF 442 and associated convolution operation 444 generating the blurry signal 432).

In the context of optical image analysis (FIG. 4B), the point spread function (PSF) 442 describes the response of an imaging system to a point source or point object and may be referred to as an impulse response where the PSF 442 represents the impulse response of an in-focus optical system. The PSF 442 can therefore represent the component of an image representing an unresolved object and is the time domain version of the transfer function of the imaging system. Such PSFs are applicable to Fourier optics, electron microscopy and other imaging techniques including confocal and fluorescence microscopy. The image of a complex object is therefore a convolution of the actual object and the PSF.

According to the present disclosure, non-optical sequencing data may be modelled using PSFs and resolved by blind deconvolution methods as will be described in greater detail hereinbelow. In various embodiments, representation of sequencing data in this manner provides a novel approach to sequence signal resolution that may provide substantial improvements, both in terms of basecalling accuracy and computational efficiency over conventional probabilistic and machine learning basecalling methods. Such enhancements can be further realized by transforming the convolution operation 424 which occurs in the time domain into a multiplication operation in the frequency domain as shown in FIG. 4C.

As applied to non-optical sequencing signals 410 in the frequency domain, the convolution operation 424 becomes a multiplication 454. Thus, deconvolution of the observed non-optical sequencing signal 412 into the resolved non-optical sequencing signal 414 becomes a multiplication operation 454 by the inverse of the PSF 422. While it will be appreciated that a deconvolution function may be applied to reconstruct the original sequencing signal as shown in FIG. 4A such an approach is computationally complex and may take considerably longer than deconvolution of sequencing signal data in the frequency domain. Notably, applying the signal processing approach illustrated in FIG. 4C in the frequency domain, desirably markedly reduces the complexity of obtaining a resolved signal 414 from the observed or detected sequencing signal 412. Those of skill in the art, will appreciate that processing a frequency domain-based solution as described above may result in arriving at an accurate representation or solution more rapidly.

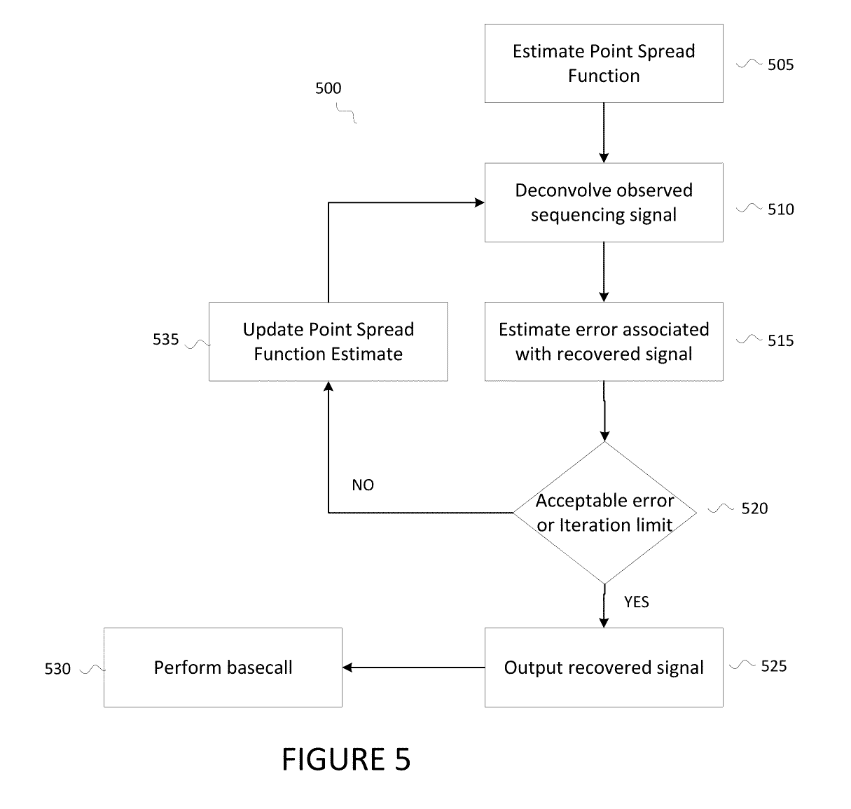

FIG. 5 illustrates an exemplary method for blind deconvolution and dephasing of sequencing data using the frequency domain-based signal deconvolution approach discussed above. In various embodiments, the method 500 provides for iterative sequencing signal error estimation where an initial Point Spread Function (PSF) corresponding to the observed sequencing signal is estimated according to step 505. The PSF may be viewed as an estimate of phasing effects upon the sequencing signal. During stepwise sequencing-by-synthesis, a population of sequencing templates associated with a sample fragment (for example clonal templates on a selected bead, particle, or contained within a well or template colony) may be subject to phasing effects. Phasing effects result in a portion of the templates being sequenced for a given sample template becoming out of sync with respect to one another. Phasing effects may further be characterized as the effects of carry forward and/or incomplete extension. Such phasing effects, may reduce the overall signal intensity for selected sequencing signal and further spread the signal over one or more reagent flows. In various aspects, the net results of these phasing effects is to obscure or “blur” the observed sequencing signal making downstream basecalling more difficult to perform and/or may reduce the confidence in the overall basecall. By deconvolving the observed signal applying various PSFs, a recovered sequencing signal may be obtained that enhances or improves basecalling accuracy. Thus, in step 510, the observed (e.g. raw) sequencing signal is deconvolved where the inverse of the estimated initial PSF multiplied by the observed sequencing signal generates a recovered (e.g. resolved) sequencing signal. As discussed above, such operations are computationally efficient and may be performed in parallel for large quantities of sequencing signal data. Further, such approaches may be adapted for use in connection with configurable hardware-embedded processors such as FPGAs and GPUs for high throughput computational execution.

In accordance with the present disclosure, signal deconvolution may be conducted using a Wiener deconvolution approach. This method is well suited to address the type of noise and phase-synchrony issues present in nucleotide sequencing data. In principal, a Wiener filter may compute a statistical estimate of an unknown signal (e.g. recovered sequencing signal) using a related signal (e.g. observed sequencing signal) as an input. The process attempts to apply a filter (e.g. PSF) to the known signal to produce the estimated or recovered signal as an output. In one exemplary approach, the known signal may comprise the unknown/recovered signal of interest that has been corrupted or “blurred” by additive noise (e.g. systematic errors and phasic synchrony loss). The Wiener filter generates a signal approximation for the recovered signal for which these noise components have been filtered out.

In the context of deconvolution of nucleotide signal data, the Wiener deconvolution method may apply a filter based on the PSF to transform the observed sequencing signal data. The Wiener filter may further attempt to minimize the mean square error between the observed nucleotide signal and the desired resolved nucleotide signal. In the context of the present disclosure, the Wiener deconvolution approach may process the nucleotide sequencing signals by operation in the frequency domain attempting to minimize or reduce the impact of deconvolved noise at frequencies which have a poor signal-to-noise ratio.

One exemplary application of the Wiener deconvolution method is described according to the equations below:

y(t)=h(t)*x(t)+n(t) (Equation 1)

where y(t) represents the observed nucleotide incorporation signal associated with a selected well, target, or sample; h(t) represents an input PSF (for example that takes phase synchrony errors and/or other systematic errors into account); * is the convolution operator; x(t) represents an idealized, predicted, or resolved nucleotide incorporation signal for a selected nucleotide incorporation event; and n(t) provides an estimated or predicted noise contribution component.

Wiener deconvolution may proceed according to:

\hat{x}(t)=g(t)*y(t) (Equation 2)

where \hat{x}(t) represents an estimated idealized or resolved incorporation event; g(t) represents the Wiener deconvolution function; * is the convolution operator; and y(t) represent the observed nucleotide incorporation signal associated with a selected well, target, or sample.

As discussed above, a generalized solution may be simplified by operation in the frequency domain where:

\hat{X}(f)=G(f)×Y(f) (Equation 3)

such that:

$$G ( f ) = \frac{{H}^{*} ( f )}{{\vert H ( f ) \vert}^{2} + \frac{N ( f )}{S ( f )}} \qquad ( Equation 4 )$$

where H*(f) represents the complex conjugate of the frequency domain representation of the PSF and

$$\frac{N ( f )}{S ( f )}$$

represents a reciprocal approximation of the signal to noise ratio spectrum. In one exemplary consideration, the term

$$\frac{N ( f )}{S ( f )}$$

may be substituted with a noise term σ². The noise term may further be calculated during the above-indicated normalization processes. Additionally, for simplicity, some components of noise can be assumed as additive white Gaussian noise (AWGN) to approximate background noise for the sequencing signals under evaluation.

In various embodiments, the PSF is estimated to solve the Wiener Deconvolution problem:

X̅(f)=G(f)×Y(f) (Equation 5)

Solving for:

$$G ( f ) = \frac{{H}^{*} ( f )}{{\vert H ( f ) \vert}^{2} + \frac{N ( f )}{S ( f )}} \qquad ( Equation 6 )$$

Where X̅(f) is the frequency domain representation of the ideal homopolymer sequence, G(f) is the frequency domain representation of the deconvolution kernel, Y(f) is the frequency domain representation of the observed flow sequence, and

$$\frac{N ( f )}{S ( f )}$$

is the inverse or the noise spectrum. In one exemplary embodiment,

$$\frac{N ( f )}{S ( f )}$$

may be substituted with the observed standard deviation of the zeromer noise σ² calculated during the well normalization process. Such an approach may attribute noise profile characteristics as additive white Gaussian (AWGN). In various embodiments, the initial kernel may commence as an impulse that assumes an initial nucleotide flow is not subject to significant phasing effects. Correspondingly, a window size may be associated with a single flow thereby matching the PSF kernel size.

According to the present disclosure, the PSF may be used to deconvolve a selected window of nucleotide flows under consideration. Further, the PSF may be used to estimate the corresponding homopolymers where the window of flows may match or correspond to the kernel length. Integer or idealized representations for homopolymers may be convolved with the PSF to estimate the raw data signal. Using this information, the difference between an observed raw signal and an estimated raw signal in the time domain may correspond to the noise and contributing PSF error.

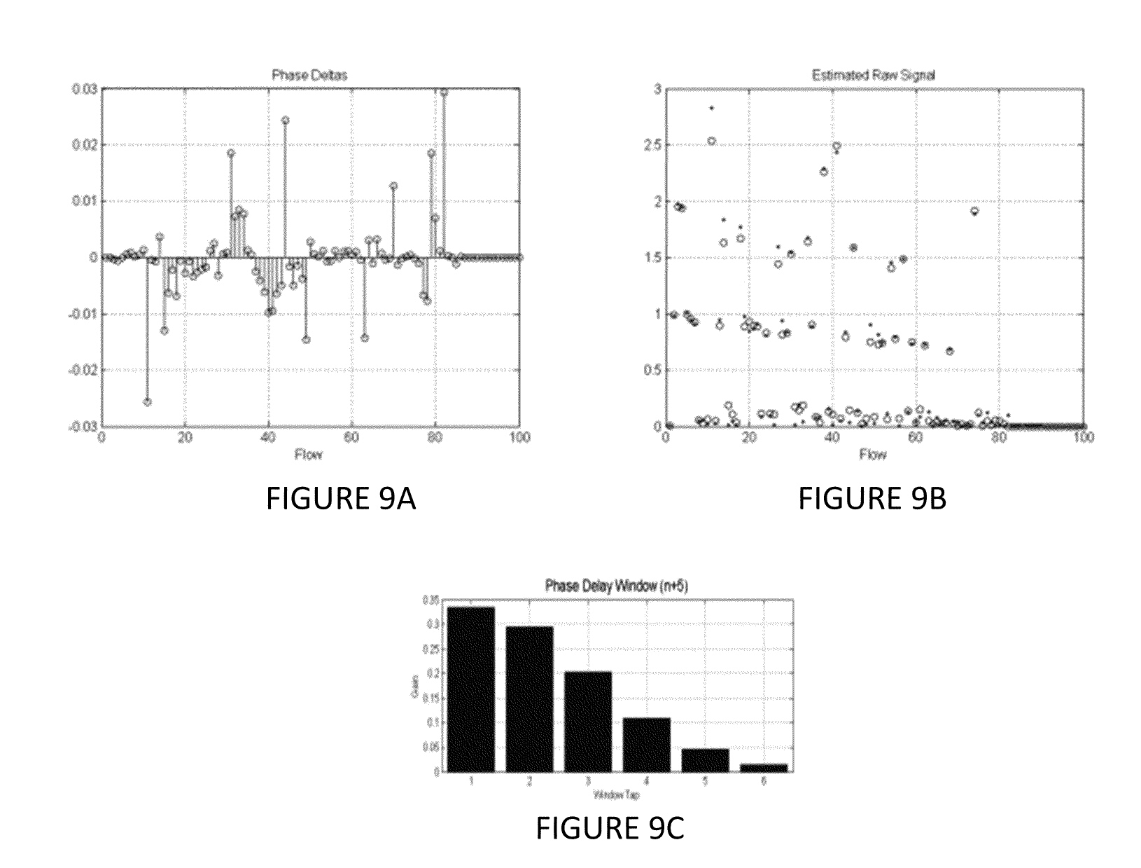

As will be discussed in greater detail hereinbelow, where elements of a difference vector associated with the observed raw signal and the estimated raw signal are of greater magnitude than the expected noise value σ², the PSF may be iteratively updated by subtracting or removing a portion or component of a corresponding error term. A boundary or range to such iterative processing may be set according to a selected iteration limit where the PSF may be refined and/or updated until the iteration limit is reached. Such as process may further be repeated until a portion of or substantially all elements of the difference vector are expressed with a magnitude not greater than the expected noise value σ². In various embodiments, stepwise errors in the estimated raw signal may occur and be accounted for. For example, a stepwise error may be identifiable as a location or point at which a selected PSF exhibits a change in behavior. Such behavior changes may result in the PSF deviating from providing relatively accurate estimations of a selected raw signal to relatively poor or low confidence estimates of the selected raw signal. Deviations in estimation may be due, at least in part, to changes or perturbations in phasing dynamics associated with a selected nucleotide flow or sequencing signal (Exemplified with reference to flow “11” in FIG. 9B discussed below).

In various embodiments, convolution and deconvolution processing may desirably be performed in a frequency domain. Such an approach facilitates data analysis by simplifying evaluation of a phase associated with a selected difference vector. Additionally, changes in phasing dynamics for a selected nucleotide flow or across nucleotide flows may be detectable in the frequency domain by determining or locating a phase point having or trending towards a magnitude greater than a selected variance a (Exemplified with reference to flow “11” in FIG. 9A discussed below). At such a point or position, the PSF may be updated, for example, by convolving the existing PSF with selected a phase delay (e.g. incomplete extension) and/or phase advance (e.g. carry forward) component or kernel. Such an approach may add at least one delay element and/or at least one advance element to the PSF. Thereafter, an updated kernel may be re-fitted to the nucleotide sequence data and further evaluated. These processes may result in one or more of the new introduced elements being removed as appropriate or desired. It will further be appreciated that the phase advance and the phase delay components of the PSF may be constructed and/or evaluated separately.

In one example, a peak associated with a selected PSF may be defined as a sum of a previous PSF from which a difference value is taken. The difference value may further be representative of the sum of one or more selected phase advance and/or phase delay elements. Such elements may be obtained from the currently iterated PSF multiplied by or applying selected droop and/or gain factors. In various embodiments, the PSF may be truncated, for example by evaluation of its values, to become sufficiently small to limit computational complexity. The PSF may also be constructed or estimated in a time-domain and subsequently converted to a frequency domain using various transform techniques. Exemplary transformation techniques include but are not limited to discrete Fourier transform (DFT) analysis and fast Fourier transform (FFT) analysis. In various embodiments, a local gain slope may be estimated from the difference vector by taking the ratio of the zeromer errors divided by the difference between zeromer and onemer errors and subsequently calculating or determining a vector cross-product applying a normalized Gaussian half-window of approximately equal length to the resulting gain vector (Exemplified in FIG. 9C).

In various embodiments, PSF estimation may be performed using a phasing matrix. The phasing matrix maps known, expected, or predicted phase synchrony effects such as carry forward, incomplete extension and droop that may arise during a sequencing reaction or run. The phasing matrix may be developed using training data or insights gained from previous sequencing datasets. In various embodiments, the phasing matrix generation process may include pre-calculating a component common to the matrix calculations such that computation load or demands are reduced during subsequence parameter estimation processes. Additionally, rather than calculating all possible values associated with the phasing matrix, windowing techniques may be used to calculate the most significant area of the matrix that will affect downstream calculations while less significant terms outside of the window may be deprioritized or ignored further reducing computational complexity and demand. In various embodiments, the phasing matrix is calculated or determined once per phasing window and may contain PSFs for some or all of the nucleotide flows within a selected phasing window.

According to various aspects of the present disclosure, a two dimensional deconvolution model may be applied to deconvolve the observed sequencing signal. Such an approach may be desirable where irregular flows or non-serial flows are using during nucleic acid sequencing. Serial nucleotide flows typically include a cycle of repeating nucleotide flows such as “AGTC” “GACT” “TCGA” or “GCAT” or other pattern where each nucleotide is introduced in a stepwise manner without re-introducing a particular nucleotide until all other nucleotides have been introduced. While such serial nucleotide flow patterns are used on many sequencing platforms, some technologies employ non-repetitive flow ordering during the sequencing process. Non-repetitive flow orderings, may include a non-serial characteristic where a selected nucleotide is reintroduced two or more times before all nucleotides have be introduced in the sequencing reaction. Non-serial flow orderings advantageously used to reduce phasing effects and aid in correction of phase errors during sequencing to improve sequencing accuracy and overall read lengths. An exemplary non-serial flow ordering may comprise a 32 base sequence such as “TACGTACGTCTGAGCATCGATCGATGTACAGC”, however, many other non-serial flow orderings may be applied in the context of nucleic acid sequencing. Additional details of non-serial flow orderings and phase correction are described in U.S. patent application Ser. No. 13/157,865 (US Patent Pub # US20120035062) to Schultz J, et. al. which is incorporated by reference in its entirety.

Whereas a linear deconvolution model can be applied to serial flow orderings described above, non-serial flow orderings, by virtue of their irregular sample order may desirably employ a different deconvolution model including the aforementioned two dimensional model. In various embodiments, the two dimensional deconvolution model permits each nucleotide in the flow order to be separately considered as a linear sequence of samples. Nucleotide flows and PSFs may further be mapped into a matrix (for example a two dimensional matrix) and PSF peak locations noted for subsequent consideration. In various embodiments, the output of the deconvolution process may provide an estimate for some or all of the points or values in the matrix applying the PSF for the current, selected, or desired flow.

In Step 515, an estimate for the error for the recovered signal is generated and the error estimate evaluated in step 520 to determine if further error iteration will be performed. In various embodiments, if the error associated with the recovered sequencing signal is sufficiently low then the method will proceed to output results including the recovered sequencing signal. The recovered signal may then be used in downstream basecalling operations in step 530 where the degree of confidence in making an accurate basecall is improved using the recovered sequencing signal versus the observed sequencing signal due at least in part to the reduced error (analogous to a sharper recovered image relative to a blurred observed image).

Where the error associated with the recovered sequencing signal has not achieved a desired degree of reduction, the method 500 may perform one or more additional iterations proceeding to update the PSF in step 535. The updated PSF is then applied to deconvolve the observed sequencing signal in step 510 and process repeated until an acceptable error achieved. In various embodiments, an iteration limit or threshold may be used as criteria for evaluation in step 520 to output a recovered signal following a preset or desired number of iterations of the method 500. Such an approach may desirably avoid performing large numbers of iterations on sequencing data where the improvement in quality of the recovered signal becomes relatively small or does not substantially improve the accuracy with which subsequent basecalling may be performed. In various instances, low quality signal data, data containing significant errors or artifacts, or data that cannot be not readily accounted for by the estimated PSFs passes through the methods by applying one or more data filters and/or setting an iteration limit to avoid unnecessary or lengthy cycling on the sequencing data which may not significantly improve the quality of the recovered signal. The iterative aspect of the method can be flexibly configured to repeat until observed error is within an expected or tolerated range, preset to a desired number of iterations or terminated when an upper bound iteration limit is reached.

As discussed above, the PSF applied to the observed sequencing signal may attempt to estimate the contribution or effect of phasing that results in a lower quality observed sequencing signal relative to the idealized resolved sequencing signal. In one exemplary approach, PSF estimation for non-optical sequencing signals may be analogized to an optical image where one or more nucleotide flows for a sequencing reaction may be associated with a well or sample containment region referred to as a “pixel”. A series of flows may then be associated with the “pixel” forming a 1×n “image”. According to this model, the PSF commences as an impulse at the first flow and continues to develop or evolve over the course of the series of various nucleotide flows during the sequencing process. As downstream nucleotide flows are subject to the errors and perturbations of predecessor nucleotide flows, the PSF from flow “n” may be used as a starting estimate for the next flow “n+1” and subsequent flows thereafter. This approach is consistent with the phasing process where sequencing of clonal templates results in gradual and/or cumulative increases in error as sequencing proceeds through extended read lengths.

Each nucleotide flow may be considered separately in parallel with other nucleotide flows. A separate PSF may also be applied for each nucleotide flow such that the behavior of the various nucleotides is not necessarily considered equivalent. Changes to the PSF as nucleotide sequencing proceeds through the length of the read may further be determined on an event driven basis rather than on the assumption that each of the events are of the same or similar magnitude. PSF updating or re-estimation need not occur with each iteration but rather updated only as applied to selected situations. For example, a selected PSF may elected not to be updated where the PSF does not adequately describe the behavior of the current nucleotide flow under consideration. It will be appreciated that the PSF estimation may take into account more than one error mode or factor separately and independently from one another. For example, the PSF estimation of phase advance (e.g. carry forward) and phase delay (e.g. incomplete extension) may be processed as separate and independent events or phenomenon and consequently estimated PSFs may take into account each of this factors separately or in a combined manner.

In various embodiments, the iterative process described above in connection with FIG. 5 may include a normalization step. For example, a two-stage normalization approach may be applied where in a first stage per nucleotide regional gain and offset are estimated from early nucleotides flows such as for known “key” flows which typically initiate the template sequencing process. Estimates may be selected for some or all of the flows associated with a region of wells or sample containment areas that may be expected to exhibit similar behaviors in terms of phasing effects. These estimates may then be applied during a second stage normalization mode where well-specific effects are taken into consideration.

For example, “total energy normalization” processing may be performed where the assumption that all nucleotides behave similarly or identically is relaxed. In various embodiments, the assumption that the behavior of the nucleotide flows during the key flows are near perfect is relaxed or discarded for a total energy normalization process. Additionally, the behavior of zeromers (nucleotides flows that do not result in an incorporation event) may be similarly relaxed such that some amount of signal is tolerated in a nucleotide flow associated with or expected to be a zeromer. Total energy normalization may further extend the analysis beyond the principal key flows into the expected sample template sequencing flows. Such total energy normalization may extend out for a portion of or the totality of a selected nucleotide flow order (for example a full nucleotide flow cycle of 8, 16, 32, or more flows). In the case of extending for a selected number of flows (for example the first 32 flows), under the modified total energy normalization approach the first 32 flows may be assumed to be similarly behave and not subject to significant signal degradation by phasing effects.

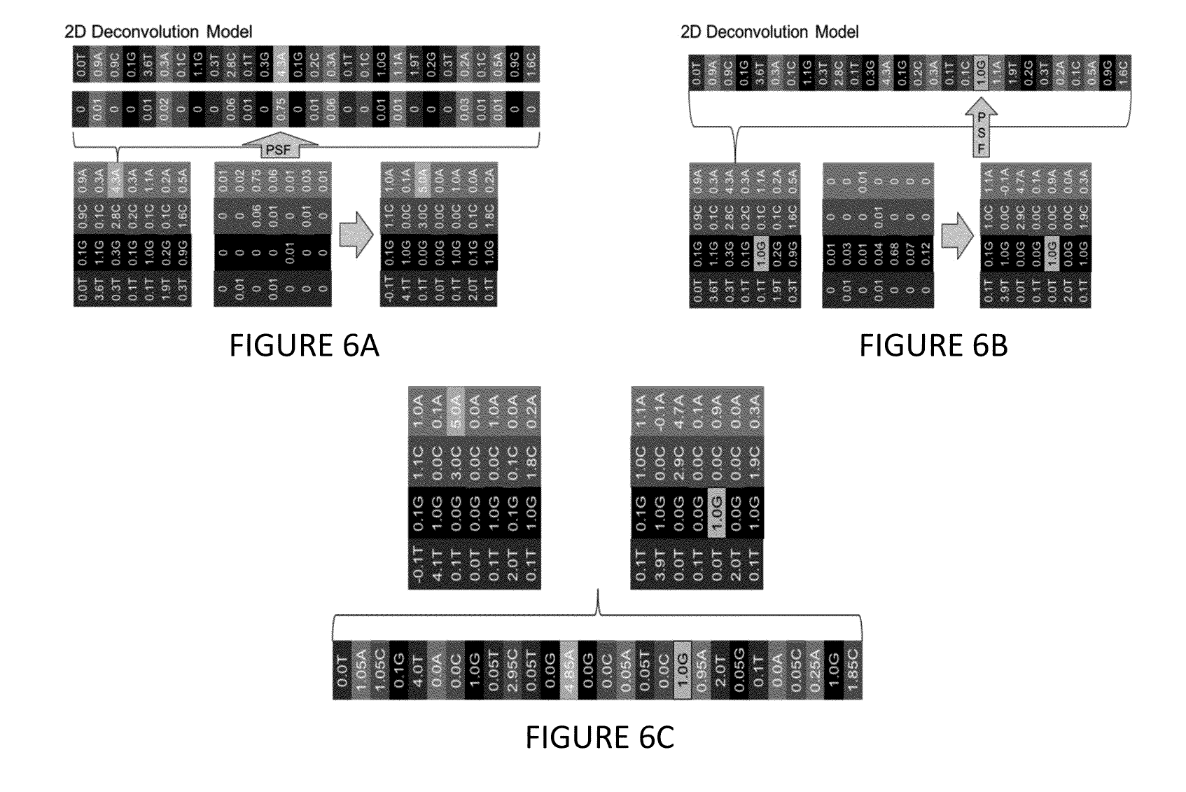

FIG. 6A illustrates an exemplary PSF and associated window of selected raw or observed data that may be re-organized or transformed from a selected non-cyclic linear flow order into a 2D matrix of associated flows for a nominated or candidate target flow. In the example, each row of the matrix may be representative of a single nucleotide type and each column representative of an associated flow number for the corresponding nucleotide. Arranging the data in this manner may be desirable to impart an ordering to which a 2D FFT may further be applied for deconvolution processing. The resulting matrix then provides a representation of dephased signal data.

FIG. 6B applies a similar approach as FIG. 6A as demonstrated for a different selected flow of a different nucleotide within a selected window. FIG. 6C depicts a transformation of resulting dephased data from FIGS. 6A and 6B re-organized back into a substantially linear sequence as provided originally. In various embodiments, such an approach may be accomplished by determining a mean or average associated with the dephasing results from each flow thereby reducing potential effects or variance from outlier data. Such a process can further be repeated for one or more flows within a selected window that is detected or determined to be associated with an incorporation event. Additionally, these processes can be repeated where a determination is made to perform a PSF iteration.

In various embodiments, per nucleotide gain values may be calculated as the mean of the key flow onemers for each nucleotide after standard key normalization has been applied. Per nucleotide offset values may further be calculated as the mean of the key flow zeromers for each nucleotide after standard key normalization has been applied. A noise approximation (σ²) may further be calculated from the standard key normalization zeromer key flows.

An exemplary approach to determining well specific tuning parameters is described by the equation below. While the parameters are discussed in connection with a particular nucleotide flow (in this case an “A” flow) it will be appreciated that the methodology can be applied to other nucleotide flows of different compositions.

$$local ( vecA ) = \frac{norm ( vecA ) - offset ( A )}{gain ( A )} \qquad ( Equation 7 )$$

Here, vecA may correspond to a vector of dNTA flows; norm(vecA) may correspond to vecA data following normalization; offset(A) may correspond to a scalar value determined for example from a selected zeromer of norm(vecA) identified or associated with key flows (e.g. initial nucleotide flows and associated signals obtained during the sequencing run); and gain(A) may correspond to a scalar value obtained or represented by taking a onemer associated with norm(vecA) during the selected key flow(s). A resultant vector local(vecA) may contain dNTA values associated with a selected well that is normalized such that key flows may have substantially zero offset and/or unity gain.

According to the methods, negative values in local(vecA) may be replaced with zero such that for an exemplary flow range (between 1 and 8 as shown) the equation below applies:

$$tuneGain ( A ) = \frac{\sum local ( vecA [ 1 \text{:} 8 ] )}{\sum round ( local ( vecA [ 1 \text{:} 8 ] ) )} \qquad ( Equation 8 )$$

By way of example, a sum of the first eight locally key normalized dNTA flows may be divided by a sum of integer approximations for those flows. During early rounds of sequencing or near the start of the template sequencing process phasing effects may be expected to be relatively small and/or insignificant. In some instances, these minor phasing effects or contributions may be of a magnitude where rounding will yield a correct homopolymer call. In various embodiments, accumulating or determining an energy (as represented by signal values) in some or substantially all of the flows may provide a scalar dephased gain value, tuneGain(A), to be determined according to the equation:

$$tuned ( vecA ) = \frac{local ( vecA )}{tuneGain ( A )} \qquad ( Equation 9 )$$

Here, data may be scaled by locally key normalized dNTA flows applying a total energy gain value, tuneGain(A) and providing total energy normalized dNTA values. Such values may then be used for dephasing analysis. The steps described above can then be repeated for each nucleotide.

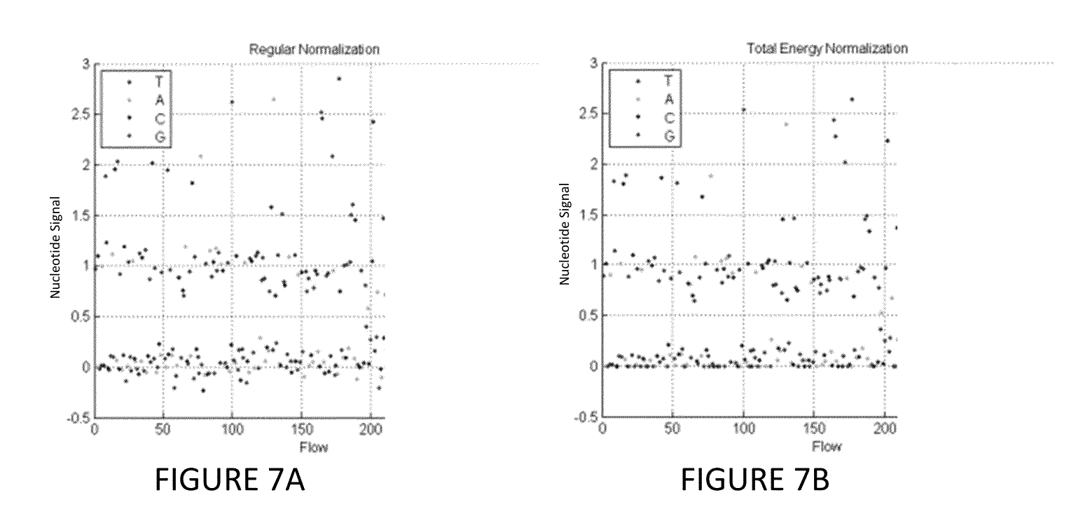

An illustration of the results of total energy normalization according to the exemplary approach described above is depicted in FIGS. 7A-B. As shown in FIG. 7A, nucleotide signals (y-axis) for sequential nucleotide flows (x-axis) during an exemplary sequencing reaction are plotted. For a selected nucleotide flow, basecalls are determined at least in part by the approximate or closest integer value (0, 1, 2, 3, etc.) to which a nucleotide flow signal resides. For example, nucleotide flow signals having a value of 1 are likely indicative of “1-mers” where a single nucleotide incorporation event is predicted to have taken place. With knowledge of the particular nucleotide associated with the selected flow number, an expected basecall can thus be discerned from the data. In the case of a nucleotide flow signal having a value of 2, such a flow signal is associated with a “2-mer” representative of a homopolymer having 2 same species nucleotides (e.g. “AA”, “GG”, “TT”, “CC”) located next to one another in the nucleotide sequence. In the case of a nucleotide flow signal having a value of 0, such a flow signal is associated with a “0-mer” representative of a non-incorporation event where for a selected flow no nucleotide was incorporated. Thus for “0-mer” flows, it is predicted that the sequence of the sample template strand being sequenced does not provide a complementary match to the current nucleotide being introduced in the nucleotide flow. By assembling the identified nucleotide signals based on the nucleotide flow order, the expected sequence of the sample template strand may be discerned.

As shown in FIG. 7A, nucleotide signals do not necessarily reside exactly along or equate to discrete integer values. Basecalling in association with nucleotide flows where the nucleotide signal is not an exact integer value may proceed by evaluation of where the nucleotide signal resides with respect to the closest integer values. Thus a basecall residing close to the whole number integer “1” versus the whole number integer “2” may be associated with a “1-mer” call for the particular nucleotide associated with the flow. In instances where the nucleotide signal is not closely associated with a particular whole integer value, some amount of uncertainty as to the appropriate basecall or homopolymer status may exist. For example, in FIG. 7A nucleotide signals that reside generally evenly between “1” and “2” or “0” and “1” may be more difficult to call with certainty.

In general, normalization of the sequencing signal data attempts to improve the signal distribution and reduce variance and signals residing between two whole integer states. As shown in FIG. 7B, applying the total energy normalization methods described above may improve the distribution of nucleotide signals such that they tend to cluster or are more tightly grouped around whole integer values. Clustering or grouping in this manner improves the accuracy and confidence in the basecall being made. Thus, in comparing the resultant nucleotide signals for the regular normalization and the total energy normalization approaches across the nucleotide flows 1-200, a general trend towards tighter clustering around whole integer values is observed. From this data, those of skill in the art will appreciate that the total energy normalization methods may therefore give rise to higher accuracy data.

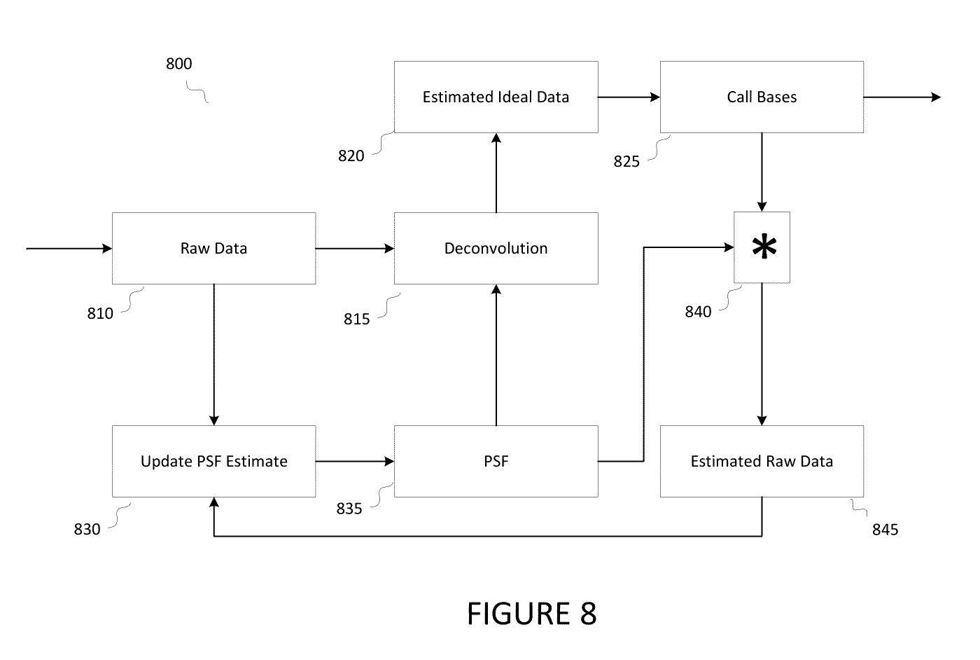

FIG. 8 further illustrates an exemplary method for nucleotide sequencing signal processing that may be used in connection with the above-described Wiener deconvolution methods. Input raw sequencing signals are received in state 810. Such data can be received and processed directly on-instrument or off-instrument and further may include various pre-processing steps including normalization, noise reduction, signal trimming and data outlier removal, and other operations. Deconvolution of the sequencing signal data proceeds in state 815 using a selected PSF determined, for example, according to the methods described above. The resulting estimated, idealized, or resolved sequencing signal data generated in state 820 may then be used for basecalling in state 825. Where the basecalling operations generate sufficiently high quality or high confidence basecalls the method may output the basecalls directly. However, in many instances it may be desirable to iterate or refine the estimated or resolved sequencing signal data further.

For iterative operations, input raw sequencing signals received in state 810 may be used to update or refine the PSF according to the characteristics of the data and signals in state 830. The updated PSF may then be applied in state 835 and further used in connection with basecall estimates generated in state 825 in a convolution operation 840 to generate an update or refined estimate of the raw (e.g. observed) sequencing signal data in state 845. The updated estimated raw sequencing data may further be applied in state 830 to update the PSF estimate. Taken together, the various states and processes may iterate for a fixed number of cycles or until a desired degree of data quality or accuracy is achieved in the output basecall result.

FIGS. 9A-B illustrate metrics for resulting from operation of the aforementioned methods on an exemplary sequencing signal dataset. As discussed above, the original or resolved sequencing signal may be estimated by applying a per-flow PSF to the deconvolved signal data. In various embodiments, phasing events may be detected by comparing differences in a Fast Fourier Transform (FFT) phase of the original/resolved signal with a FFT phase of the estimation of the original/resolved signal further scaled by the difference between the original and estimated signals. As will be appreciated by one of skill in the art, fast Fourier transform (FFT) analysis computes the discrete Fourier transform (DFT) of a sequence, or its inverse. Fourier analysis may therefore convert a sequencing signal from its original domain (for example in time or space) to a representation in the frequency domain and vice versa. According to various approaches, FFT analysis may be applied to sequencing signal data to rapidly compute transformations by factorizing the DFT matrix into a product of sparse (mostly zero) factors. Consequently, application of the FFT to the sequencing signal data may help reduce the computational complexity for computing the DFT.

Phase deltas (FIG. 9A) are shown for an exemplary sequencing signal dataset (FIG. 9B). According the methods described herein, significant noise variance (o) may be considered as phasing events where the PSF may be desirably iterated to improve the quality of the data and the resultant basecall. Where noise variance is below a selected threshold, the basecall quality may inferred to be acceptable without further PSF iterations. As shown, flows with notable high phase variance that exceed a selected threshold or are or outside of the mean range of other signal data may be further iterated and refined to improve basecall accuracy.

In various embodiments, a selected PSF may be iterated by separately testing for phase delay (e.g. incomplete extension/negative phasing) and phase advance (e.g. carry forward/positive phasing) events. A test kernel may further define the order and relative proportion of the phasing model function contributions. In various embodiments, a first order model may be used, however, it is contemplated that other models may be similarly applied. The exact value of the test kernel need not be critical where each phasing event is scaled proportionally.

In an example, the phase delay (or advance) at a current selected flow may have the relevant test kernel added to the PSF and a surrounding window or selected number of flows processed by deconvolution. In various embodiments, those flows following the current flow are considered for phase delay and flows preceding the current flow are considered for phase advance. The estimated raw signal deltas within a selected window may be compared with the estimated raw signal deltas from before the test kernel was added to the PSF. From this analysis, a gain slope (scaled by selected elements within the window) may be estimated and the PSF may be updated with a scaled version of the test kernel.

An exemplary application of the method described above is set forth by the equations below and further discussed in connection with FIG. 9C:

ZeroErr=raw[win]−est[win] (Equation 10)

The equation above provides a method by which to calculate a vector of zeromer errors within a selected window subtracting or removing estimated zeromers from observed zeromer values.

OneErr=raw[win]−est[win] (Equation 11)

The equation above provides a method by which to calculate a vector of onemer errors within a selected window subtracting or removing estimated onemers from observed onemer values.

$$GainVec = \frac{ZeroErr}{ZeroErr - OneErr} \qquad ( Equation 12 )$$

The equation above provides for OneErr vector trimming to match the length of the ZeroErr vector. The resulting ratio between zeromer errors and the difference of the zeromer and onemer errors may then be used to generate a vector of gain errors.

In various embodiments, removal of outlier data may be desirable. Such outlier data may include non-applicable nucleotide (NaN). Removal of outlier data may arise where selected OneErr values are very similar or equivalent to corresponding ZeroErr values. Such data may further cause singularities or large errors and thus removal of these values may be desirable to help provide integrity in subsequent calculations.

gain=GainVec×WinVec (Equation 13)

According to the equation above, a final scalar gain value may then be applied to a “generic” or selected test kernel during PSF iteration. In various embodiments, application to the test kernel may be performed by applying a “clean” GainVec vector (with outliers and NaN's removed) and computing a vector cross product with a Gaussian half-window (WinVec) of approximately equal length (depicted in FIG. 9C). This approach and window may reduce the significance of contributions from flow elements further away from the current flow.

In various embodiments, a PSF constructed in the time domain may be converted to the frequency domain using FFT analysis. The phase delay and phase advance components of the sequencing signal may further be constructed separately. Where a phase delay event is identified for a current or selected flow, the PSF may be iterated by convolution with a scaled version of the phase delay test kernel. Where a phase advance event is identified for the current or selected flow, the PSF may be iterated by convolution with a scaled version of the phase advance test kernel. In an exemplary application, the peak term of the PSF may be defined as the sum of the previous PSF minus the sum of the phase advance and phase delay elements of the iterated PSF. This term may further be multiplied by a droop gain factor, as calculated above.

A further example of the blind deconvolution methods described for sequencing signals may comprise normalization of a selected sequencing signal dataset using estimations of noise components or signal contributions (σ²). In various embodiments, noise estimates may be obtained for a portion of, or substantially all, wells or sample containment areas in a selected analysis region to estimate a local noise component σ².

Tracking the method described in FIG. 8, phase advance and phase delay event vectors may be initialized to zero values. An initial PSF may further be initialized to a selected impulse. h(t)=[1] Proceeding with deconvolution of the sequencing signal data a value for \hat{x}(t) may be obtained. Error estimation may then be performed to identify an associated or next phasing event followed by updating the PSF estimate based upon the calculated values. In an iterative process, deconvolution and subsequent steps may again be repeated to refine the PSF estimate based on a selected number of cycles/iterations or until a desired degree of basecall confidence is achieved.

As discussed above, an associated Wiener deconvolution filter simplifies to:

$$G ( f ) = \frac{{H}^{*} ( f )}{{\vert H ( f ) \vert}^{2} + {σ}^{2 }} \qquad ( Equation 14 )$$

In various embodiments, a separate PSF may be generated on a per flow basis where the substantial majority or entirety of the flow space data is iterated upon two or more times thereby refining associated PSF estimates. Subsequently, a FFT analysis may be used to convert a selected PSF to H(f). Flows covered by or in the range of the selected PSF may further be converted to a frequency domain using FFT analysis to obtain Y(f) which may then be applied to solve:

X̅(f)=G(f)×Y(f) (Equation 15)

In various embodiments, the data may further be converted back to its corresponding time domain representation applying an inverse fast Fourier transform (IFFT) to obtain x̅(t). Here the value at the current flow in x̅(t) is representative of the dephased value which may further be rounded to the nearest integer for purposes of base calling. Subsequently, the rounded value may be convolved with the PSF from the selected flow to estimate phasing effects which may further be accumulated across substantially all flows within a selected region or window. FFT phase may then be calculated and compared with the original signal FFT phase to identify the next phase event. In various embodiments, negative phase advance values may represent phase catch-up and may be modelled by applying a time-reversed version of the positively scaled noise kernel, accumulating the phase advance side of the PSF inwards by one step. While negative phase delay gain values may not as directly represent an underlying physical phenomenon, the phase delay side of the PSF may be pushed outwards by one step before applying the positively scaled kernel.

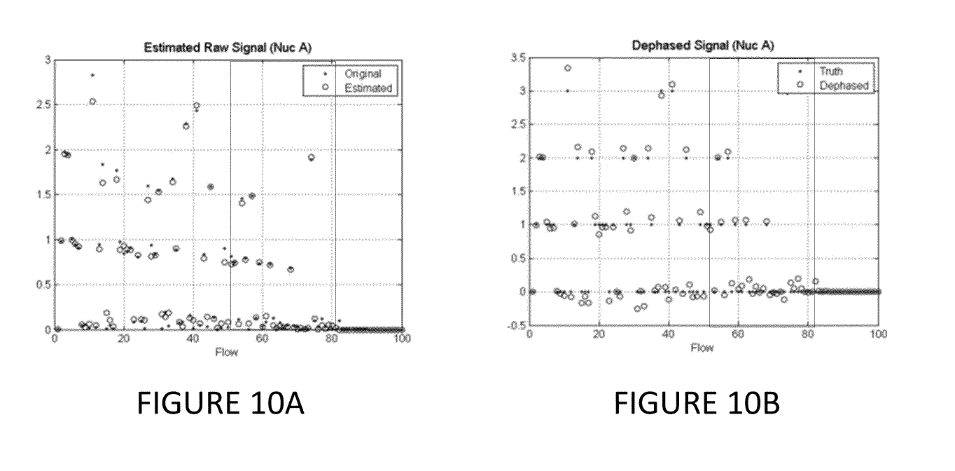

FIGS. 10A-B illustrate the application of the present blind deconvolution methods through 4 iterations of PSF estimation. Comparing the estimated raw signal data (FIG. 10A) within the selected window region versus the dephased signal data (FIG. 10B), notable improvements in data variance and tracking along discrete integer values is observable. In comparison to the estimated raw signal data, the dephased signal data processed according to the present methods may be more confidently and accurately resolved to basecalls (observe deviation in data relative to integer values for estimated and dephased signal data).

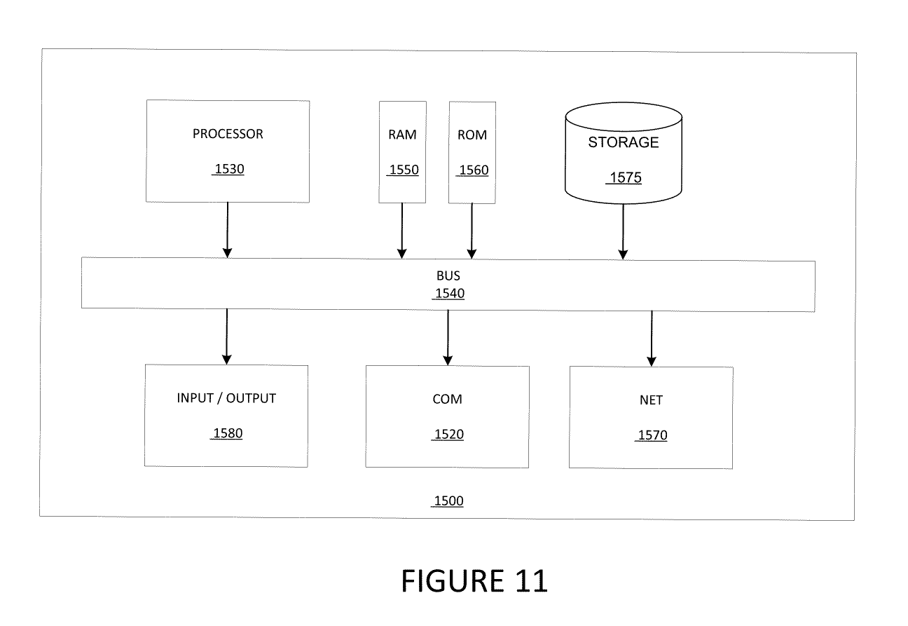

FIG. 11 is a simplified functional block diagram of a computing environment 1500 that may be configured as a computer, system, and/or server for executing the methods described above, according to an exemplary embodiment of the present disclosure. Specifically, in one embodiment, any of computers, systems, and/or servers implementing the above-described disclosure may be an assembly of hardware including, for example, a data communication interface 1520 for packet data communication. The platform may also include a processing unit, FPGA, GPU, CPU, etc. 1530, in the form of one or more processors, for executing program instructions. The platform typically includes an internal communication bus 1540, program storage 1575, and data storage for various data files to be processed and/or communicated by the platform such as RAM 1550 and ROM 1560, although the system 1500 often receives programming and data via network communications 1570. The computing environment 1500 also may include input and output ports 1580 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed application, methods, computers, servers, devices, and systems are described with exemplary reference to computer applications and to transmitting various types of data, it should be appreciated that the presently disclosed embodiments may be applicable to any environment, such as a desktop or laptop computer, etc. Also, the presently disclosed embodiments may be applicable to any type of Internet protocol that is equivalent or successor to HTTP.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for estimating numbers of nucleotide incorporations in nucleic acid sequencing-by-synthesis, comprising:
(a) disposing a plurality of template polynucleotide strands in a reaction confinement region disposed on a sensor array, the reaction confinement region containing a sequencing primer and a polymerase;
(b) exposing the template polynucleotide strands and the sequencing primer and polymerase to a series of flows of nucleotide species flowed according to a predetermined ordering of nucleotide flows;
(c) obtaining a plurality of nucleotide incorporation signals for the reaction confinement region from the sensor array, the nucleotide incorporation signals having an intensity related to a number of nucleotide incorporations having occurred in response to the nucleotide flows;
(d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and
(e) estimating a number of nucleotide incorporations having occurred for the template polynucleotide strands in the reaction confinement region based on the result of the multiplication converted back to time domain.

2. The method of claim 1, wherein iteratively estimating the point spread function representing phase advance events and phase delay comprises detecting phasing events by comparing differences in the fast Fourier transform phase of an original signal with the fast Fourier transform phase of an estimate of the original signal, scaled by a difference between the original signal and the estimate of the original signal.

3. The method of claim 1, wherein iteratively estimating the point spread function representing phase advance events and phase delay comprises constructing a phase advance component and a phase delay component separately.

4. The method of claim 3, wherein, iteratively estimating the point spread function representing phase advance events and phase delay comprises iterating the point spread function by convolving the left half of the point spread function with a scaled version of an advance test kernel if a phase advance event has been detected for a current flow.

5. The method of claim 3, wherein, iteratively estimating the point spread function representing phase advance events and phase delay comprises iterating the point spread function by convolving the right half of the point spread function with a scaled version of a delay test kernel if a phase delay event has been detected for a current flow.

6. The method of claim 1, wherein iteratively estimating a point spread function representing phase advance events and phase delay events is implemented using a field programmable gate array embedded in a sequencing instrument.

7. The method of claim 1, wherein converting the point spread function to frequency domain using a fast Fourier transform and/or converting nucleotide incorporation signals for nucleotide flows covered by the point spread function to frequency domain using a fast Fourier transform is implemented using a field programmable gate array embedded in a sequencing instrument.

8. The method of claim 1, wherein converting multiplying the frequency domain point spread function and nucleotide incorporation signals and converting the result of the multiplication back to time domain using an inverse fast Fourier transform is implemented using a field programmable gate array embedded in a sequencing instrument.

9. A non-transitory machine-readable storage medium comprising instructions which, when executed and/or implemented by a processor and/or field programmable gate array, cause the processor and/or field programmable gate array to perform a method for estimating numbers of nucleotide incorporations in nucleic acid sequencing-by-synthesis comprising:
(a) disposing a plurality of template polynucleotide strands in a reaction confinement region disposed on a sensor array, the reaction confinement region containing a sequencing primer and a polymerase;
(b) exposing the template polynucleotide strands and the sequencing primer and polymerase to a series of flows of nucleotide species flowed according to a predetermined ordering of nucleotide flows;
(c) obtaining a plurality of nucleotide incorporation signals for the reaction confinement region from the sensor array, the nucleotide incorporation signals having an intensity related to a number of nucleotide incorporations having occurred in response to the nucleotide flows;
(d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and
(e) estimating a number of nucleotide incorporations having occurred for the template polynucleotide strands in the reaction confinement region based on the result of the multiplication converted back to time domain.

10. A system, including:
a machine-readable memory; and
a processor and/or field programmable gate array configured to execute and/or implement machine-readable instructions, which, when executed and/or implemented by the processor and/or field programmable gate array, cause the system to perform a method for estimating numbers of nucleotide incorporations in nucleic acid sequencing-by-synthesis, comprising:
(a) disposing a plurality of template polynucleotide strands in a reaction confinement region disposed on a sensor array, the reaction confinement region containing a sequencing primer and a polymerase;
(b) exposing the template polynucleotide strands and the sequencing primer and polymerase to a series of flows of nucleotide species flowed according to a predetermined ordering of nucleotide flows;
(c) obtaining a plurality of nucleotide incorporation signals for the reaction confinement region from the sensor array, the nucleotide incorporation signals having an intensity related to a number of nucleotide incorporations having occurred in response to the nucleotide flows;
(d) iteratively estimating a point spread function representing phase advance events and phase delay events, converting the point spread function to frequency domain using a fast Fourier transform, converting nucleotide incorporation signals for nucleotide flows covered by the point spread function to frequency domain using a fast Fourier transform, multiplying the frequency domain point spread function and nucleotide incorporation signals, and converting the result of the multiplication back to time domain using an inverse fast Fourier transform; and
(e) estimating a number of nucleotide incorporations having occurred for the template polynucleotide strands in the reaction confinement region based on the result of the multiplication converted back to time domain.
